(19) United States
(12) Patent Application Publication
(10) Pub. No.: US 2023/0138022 A1
(43) Pub. Date: May 4, 2023

(54) METHODS OF MAKING PLURIPOTENT STEM CELLS AND USES THEREOF

(71) Applicant: NATIONAL JEWISH HEALTH, Denver, CO (US)

(72) Inventors: Max A. SEIBOLD, Parker, CO (US); Yingchun LI, Denver, CO (US)

(21) Appl. No.: 17/915,729

(22) PCT Filed: Apr. 1, 2021
(86) PCT No.: PCT/US21/25414

§ 371 Date: Sep. 29, 2022

Related U.S. Application Data
(60) Provisional application No. 63/003,661, filed on Apr. 1, 2020.
(60) Provisional application No. 63/003,670, filed on Apr. 1, 2020.

(51) Int. Cl. C12N 5/074; C12N 5/071; C12N 15/90

(52) U.S. Cl. CPC C12N 5/0696; C12N 5/0688; C12N 15/907; C12N 2501/602; C12N 2501/603; C12N 2501/604; C12N 2501/605; C12N 2501/606; C12N 2501/608; C12N 2501/727; C12N 2506/45; C12N 2506/27

(57) ABSTRACT

Disclosed herein are methods to reliably and robustly generate a pure population of airway basal cells that are capable of producing a normal mucociliary epithelium. Such basal cells may be used to treat chronic respiratory diseases, such as cystic fibrosis, chronic obstructive pulmonary disease, and asthma.

### CROSS-REFERENCE TO RELATED APPLICATIONS

This application claims the benefit of U.S. Provisional Patent Application Ser. Nos. 63/003,661 and 63/003,670, both filed on Apr. 1, 2020, the entire disclosure of each of which is incorporated herein by reference.

### BACKGROUND

Chronic airway diseases are characterized by genetically and environmentally driven dysfunction of the airway epithelium. This epithelial dysfunction includes reduction in mucociliary clearance, inflammatory immune signaling, cellular remodeling, and inadequate wound healing and homeostatic regeneration. These pathologic features are the result of aberrant regeneration and mucociliary differentiation of basal airway cells, the stem cell of the airway. This pathologic regeneration is genetic and/or acquired through environmental-programming of the basal cells. As such the production and transplant of genetically corrected basal stem cells, purged of environmental or disease-based epigenetic programming, presents a potential therapeutic strategy for chronic airway diseases. Moreover, basal stem cells generated in this manner can be used to create powerful patient-specific organoid models to study airway diseases.

De novo creation of patient-specific airway basal cells from induced pluripotent stem cells (iPSCs) may be used to produce an unlimited supply of epigenetically “pure”, and genetically manipulatable, basal cells. Although stepwise differentiation protocols have been developed which specify lung progenitors, including airway basal cells, the directed generation of a pure airway basal cell population has not been achieved. The present disclosure provides a method to reliably and robustly generate a pure population of airway basal cells capable of producing normal mucociliary epithelium. The methods disclosed herein represent a significant advance in the quest for regenerative therapeutic approaches applied to the human airway epithelium.

### BRIEF DESCRIPTION OF THE DRAWINGS

FIG. 1 shows the characterization of AEC-derived iPSCs by flow cytometry. The histograms show OCT3/4, Nanog, SSEA-4, and ALP expression in AEC donors and donor-derived iPSCs. The lighter histogram peaks represent staining with isotype control antibodies. The darker histogram peaks represent staining with indicated antibodies.

FIG. 2 shows the characterization of fibroblast-derived iPSCs by flow cytometry. The histograms show OCT3/4, Nanog, SSEA-4, and ALP expression in fibroblast donors and donor-derived iPSCs. The lighter histogram peaks represent staining with isotype control antibodies. The darker histogram peaks represent staining with indicated antibodies.

FIG. 3 shows the percentage of pluripotent marker expression by flow cytometry. Pluripotent markers (OCT3/4, Nanog, SSEA-4, and ALP) are higher in iPSCs compared to donor cells. FIG. 3 also shows that keratin (KRT 5) mean intensity change as observe by flow cytometry. Airway epithelial cells have higher keratin 5 expression compared to iPSCs.

FIG. 4 shows measurement of EPCAM gene editing efficiency as measured by flow cytometry. The lighter peak represents scramble transfected. The darker peak represents EPCAM gene edited cells.

FIG. 5 shows the cell proliferation rate after EPCAM gene editing. Cell proliferation rate was measured by trypan blue dye counting assay. Lighter bars represent scramble transfected. Darker bars represent EPCAM gee edited cells.

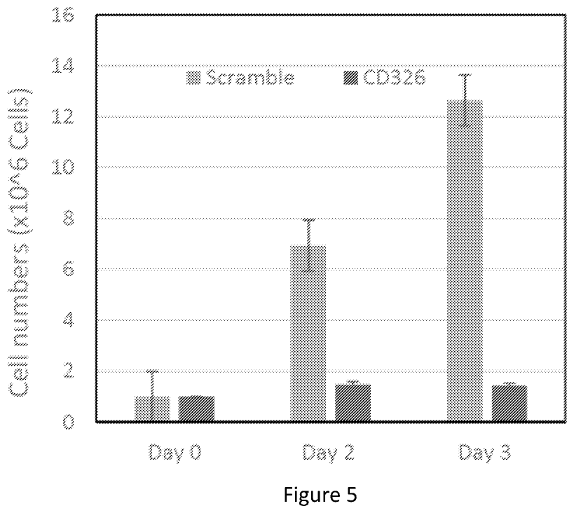

FIG. 6 illustrates a representative timeline of differentiation to functional epithelium. The Figure illustrates stage specific 2D-transwell and 3D-spheroid culture methods for producing differentiated AECs from undifferentiated iPSCs.

FIG. 7 illustrates an exemplary reprogramming protocol.

FIG. 8 shows bar plots summarizing quantification of immunolabeled cytospins from organoids (expanding), organoids (differentiating), and iBC colonies from independent iBC regeneration experiments of 5 iPSC clones. Each bar represents the average percent positive cells from a single stage of regeneration with a single iPSC clone, where error bars indicate standard deviation from n3-5 fields of view.

FIG. 9 shows that iBCs retain robust proliferative capacity, as illustrated by cumulative cent of iBCs from independent iBC regeneration experiments of 5 iPSC clones. Tissue sources for each iPSC clone are indicated in the legend.

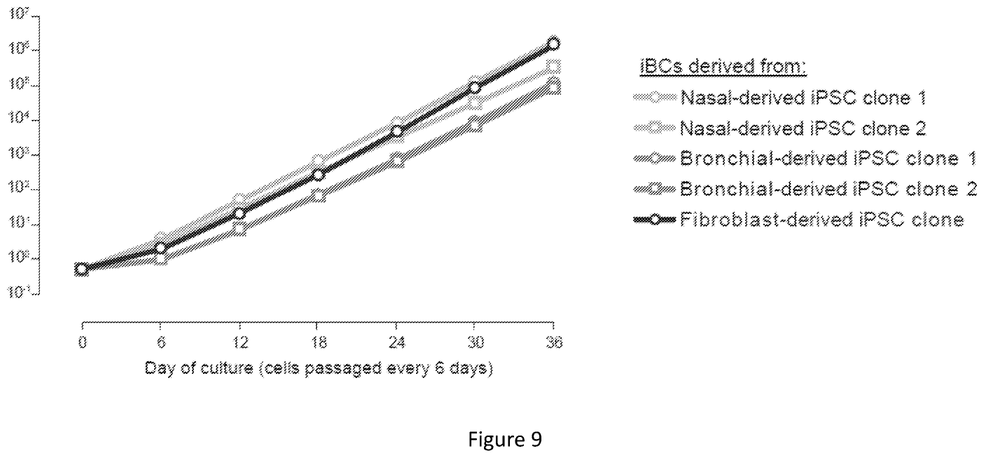

FIG. 10 shows boxplots depicting mean expression of in vitro human bronchial basal cell gene signature across organoid populations.

FIG. 11 illustrates sources of mucociliary epithelium compared to the same donor.

FIG. 12 shows H&E staining of histological sections from in vivo (FIG. 12A), in vitro (FIG. 12B), and reprogrammed airway epithelial cells from the same donor (FIGS. 12C & 12D). Scale bar is 50 μM in all images.

FIG. 13 shows quantification of primary ALI and iALI cellular composition by Area of Fluorescence (AOF) analysis, which indicates highly comparable cell type frequencies at P2, and retention of conical cellular composition in iALI over at least 5 passages.

### SUMMARY

The present application provides a virus-, DNA-, and integration-free RNA-based reprogramming method for the generation of iPSCs from nasal and bronchial airway basal epithelial cells. These iPSCs expressed pluripotency markers, exhibited unlimited proliferative potential (passaged 30 times to date), and were capable of generating all three germ layers. Existing stepwise protocols were modified (endoderm>anterior foregut>lung progenitors), directing lung progenitor organoids to differentiate into proximal airway cells. Pure airway basal cells were further specified and procured through fibroblast feeder culture (with SMAD inhibition) of these proximal airway cells. No genetic manipulation was used in this method. The induced basal cells (iBCs) generated by this method were ˜99% positive for the basal stem cell markers KRT5, TP63, and NKX2.1, and negative for vimentin. The iBCs were phenotypically identical to basal cells from which the iPSCs were derived and were passaged 7 times without loss of basal cell markers. Importantly, the iBCs can be differentiated into a highly consistent pseudostratified epithelium containing mucus secretory, ciliated, and basal cells, via standard ALI differentiation, without generating contaminating cells of other lineages.

One embodiment is a method of producing induced basal cells (iBCs), comprising obtaining induced pluripotent stem cells (iPSCs), and directing generation of iBCs from the iPSCs, wherein the process of directing lacks genetic manipulation. The process of directing may result it the production pf a homogeneous population of iBCs in which at least 80%, at least 85%, at least 90%, at least 95%, or 100% of the iBCs are KRT5⁺, TP63⁺, and NKX2.1⁺. Obtaining iPSCs may comprise obtaining, culturing, and expanding primary cells (PCs); transfecting the PCs using RNA-based reprogramming factors; and identifying and purifying iPSCs. The PCs may be obtained from an individual by methods such as cell surface brushing, surgical excision, or lavage. The PCs may be human PCs, the genomes of which may have been modified using techniques such as CRISPR. The PCs may be airway epithelial cells or fibroblasts. RNA-based reprogramming factors may comprise RNA molecules encoding octamer-binding transcription factor (Oct4) protein, Sry-box transcription factor 3 (Sox2) protein, Kruppel like factor 4 (Klf4) protein, cMyc protein, Nanog homeobox protein (Nanog), or Lin28 protein (Lin28).

Directing generation of iBCs from iPSCs may comprise forming lung organoids from the iPSCs, differentiating the lung organoids to form airway epithelial spheroids comprising airway epithelial cells, and culturing the airway epithelial cells with fibroblasts, which may be gamma-irradiated, to form iBCs. Culturing airway epithelial cells with fibroblasts may comprise Duel-SMAD inhibition and/or an inhibitor of rho-associated coiled coil containing kinase (ROCK). Forming lung organoids may comprise directing iPSCs to differentiate into definitive endoderm cells (DECs), which may express CD184 (C-X-C chemokine receptor type 4 (CXCR4) and/or CD177 (tyrosine kinase Kit protein (c-KIT); differentiating the DECs to differentiate into anterior foregut endoderm (AFE), which may express FOXA2 and/or SOX2; directing AFE cells to differentiate into Ventralized-AFE containing lung progenitor cells that are FOXA2⁺, SOX2⁺, and NKX2.1⁺; optionally enriching the population of lung progenitor cells, and culturing the lung progenitor cells to form lung organoids. Forming lung organoids may comprise 3D organoid culture.

One embodiment is an iBC producing according to the disclosed methods. The iBC may be a human iBC. The iBC may have been produced using an airway epithelial cell or a fibroblast as the PC.

One embodiment is a method of producing an epithelial tissue, comprising culturing an iBC produced according to the disclosed methods. Culturing may comprise an air-liquid interface culture.

One embodiment is an epithelial tissue produced using a method comprising, culturing an iBC produced in an air-liquid interface culture.

One embodiment is a method of treating an individual in need of such treatment, comprising administering an iBC or an epithelial tissue of the disclosure. The iBC or epithelial tissue may be administered to treat the individual for a respiratory disease. Administration may comprise transplanting the iBC or the epithelial tissue into the subject's epithelium, which may be nasal epithelium, oral epithelium, pharyngeal epithelium, laryngeal epithelium, tracheal epithelium, bronchial epithelium, and/or lung epithelium.

One embodiment is use of a disclosed method in the preparation of an iBC.

One embodiment is use of an iBC produced according to a disclosed method, in the preparation of an epithelial tissue; in preparing a primary cell or tissue-based model of a disease; or in studying a biological response to a compound or an environmental stimulus.

One embodiment is use of the disclosed methods, of an iBC or epithelial tissue of the disclosure, in the preparation of a medicament or therapeutic composition for treating a respiratory disease. The respiratory disease may be a chronic respiratory disease.

One embodiment is use of the disclosed methods, of an iBC or epithelial tissue of the disclosure, in identifying a therapeutic compound. The compound may be for the treatment of a respiratory disease.

In these methods and uses, the iBC may be a human iBC. The iBC may have been produced using an airway epithelial cell or a fibroblast as the PC.

### DETAILED DESCRIPTION

Methods have been developed for do novo creation of patient specific airway basal cells from induced pluripotent stem cells (iPSCs). Such methods may produce an unlimited supply of “pure” basal cells that may be used to treat chronic airway diseases such as cystic fibrosis (CF), chronic obstructive pulmonary disease (COPD), and asthma. The methods, which do not require genetic manipulation or the use of any genetic material, utilize culture techniques that direct the iPSCs through a series of differentiation steps, resulting in final differentiation into induced basal cells (iBCs) that can further differentiate into normal mucociliary epithelium. Accordingly, a method of the present disclosure can generally be practiced by obtaining iPSCs and using culture techniques devoid of genetic manipulation and/or the addition of genetic material, to produce iBCs. Such culture techniques may comprise directing the differentiation of iPSCs into definitive endoderm, which may be directed to differentiate into anterior foregut endoderm (AFE), which may be directed to differentiated into Ventralized-AFE containing lung progenitor cells. The lung progenitor cells may be used to form lung organoids, which may be directed to differentiate into an epithelial organoid, which may be directed to form iBCs.

As used herein, the singular forms “a,” “an,” and “the” include plural referents unless the context clearly dictates otherwise. For example, a nucleic acid molecule refers to one or more nucleic acid molecules. As such, the terms “a”, “an”, “one or more” and “at least one” can be used interchangeably. Similarly, the terms “comprising”, “including” and “having” can be used interchangeably. The claims may be drafted to exclude any optional element. As such, this statement is intended to serve as antecedent basis for use of such exclusive terminology as “solely,” “only” and the like regarding the recitation of claim elements or use of a “negative” limitation.

Certain features of the disclosure, which are described in the context of separate embodiments, may also be provided in combination in a single embodiment. Conversely, various features of the disclosure, which are, for brevity, described in the context of a single embodiment, may also be provided separately or in any suitable sub-combination. All combinations of the disclosed embodiments are specifically embraced by the present disclosure and are disclosed herein just as if each and every combination was individually and explicitly disclosed. In addition, all sub-combinations are also specifically embraced by the present disclosure and are disclosed herein just as if each and every such sub-combination was individually and explicitly disclosed herein.

The present disclosure is not limited to particular embodiments described herein. The terminology used herein is not intended to be limiting.

Any publication mentioned herein are provided solely for its disclosure prior to the filing date of the present application. Nothing herein is to be construed as an admission that the present disclosure is not entitled to antedate such publication by virtue of prior disclosure. Further, the publication dates provided may be different from the actual publication dates, which may need to be independently confirmed. All publications mentioned herein are incorporated herein by reference to disclose and describe the methods and/or materials in connection with which the publications are cited.

Unless defined otherwise, all technical and scientific terms used herein have the same meaning as commonly understood by a person of skill in diagnostics. Although any methods and materials similar or equivalent to those described herein can also be used in the practice or testing of tools and assays of the disclosure, the preferred methods and materials are now described.

One embodiment is a method of producing induced basal cells (iBCs), comprising obtaining induced pluripotent stem cells (iPSCs), and using culture methods to direct guided differentiation of the iPSCs into iBCs. The terms directing guided differentiation, guided differentiation, and the like, refer to using a series of culturing (e.g., tissue culture) steps and conditions to cause a specific cell type (e.g., iPSCs) to differentiate into a desired cell type (e.g., iBCs) either directly or through series of intermediate cell types (e.g., definitive endoderm cells, lung epithelial cells, etc.). Preferably, the culture methods used to direct guided differentiation into iBCs do not use genetic manipulation or the addition of any genetic materials.

iPSCs useful for practicing the disclosed methods may be obtained from any species of animal. Examples include, but are not limited to, humans, non-human primates, such as chimpanzees, apes and other monkey species; domestic mammals (e.g., dogs and cats); laboratory animals (e.g., mice, rats guinea pigs); birds, and, bats. Similarly, the terms individual, subject, and patient are herein used interchangeably to refer to any human or non-human animal from which cells used to practice the disclosed methods are obtained. Moreover, individuals of any age or race are covered by the present disclosure.

Induced basal cells produced according to the disclosed methods express at least one basal cell stem marker selected from the group consisting of Keratin 5 (KRT5) protein, tumor protein 63 (TP63) protein, and homeobox protein Nkx-2.1 (NKX2.1). Such proteins may be referred to as stem cell marker proteins, stem cell markers, and the like. Cells expressing such markers may be referred to as being positive (+) for the marker. Thus, in one aspect, an induced basal cell produced by methods of the disclosure may be positive for at least one stem cell marker selected from the group consisting of KRT5, TP63, and NKX2.1. In one aspect, an induced basal cell produced by methods of the disclosure may be positive for KRT5, TP63, and NKX2.1. In one aspect, an induced basal cell produced by methods of the disclosure may be KRT5+, TP63+, and/or NKX2.1+. In one aspect, an induced basal cell produced by methods of the disclosure may be KRT5+, TP63+, and NKX2.1+(a.k.a., triple positive).

As used herein, a method that is performed “without genetic manipulation”, or “without the addition of genetic material”, and the like, means a method of directing differentiation of iPSCs into iBCs in which differentiation is achieved through culturing cells sunder specific conditions, and in which no isolated or recombinant genetic material is added to the cell cultures in order to drive differentiation of the cells. Genetic material refers to DNA, RNA, modified forms thereof, and combinations thereof. Isolated genetic material refers to genetic material that has been removed from its natural cellular environment. Examples of isolated genetic material include, but are not limited to, naked DNA, cDNA, plasmids, cosmids, isolated RNA, recombinant viral genetic material, or genomic genetic material, or fragments thereof, that has been removed from a cell. Accordingly, it should be understood that adding intact mammalian cells (e.g., fibroblasts) to a culture does not constitute adding genetic material. Exemplary methods of directing the differentiation of iPCSs into iBCs without the use of genetic manipulation are disclosed herein.

In one aspect, obtaining iPSCs comprises obtaining a sample of primary cells (PCs). Examples of suitable PCs include, but are not limited to, airway epithelial cells. In certain aspects, the PCs may be fibroblasts. Any method that leaves the PCs intact and viable may be used to obtain PCs. Examples of useful methods of obtaining PCs include, but are not limited to, surgical removal of tissue followed by grinding or maceration of the tissue, brushing an epithelial surface, and lavage of epithelial-lined cavities.

In one aspect, PCs may be cultured using conditions (e.g., culture plates, medium, etc.) comprising one or more extracellular matrix proteins (e.g., laminin), prior to transfection using appropriate culture conditions known to those skilled in the art. For example, if the PCs are primary airway epithelial cells (AECs), they may be cultured using a media that supports expansion of primary AECS (e.g., PNEUMACULT™-EX Plus Medium (STEMCELL TECHNOLOGIES, INC.). As a further example, if the PCs are fibroblasts, they may be cultured using Fibroblast Expansion Medium (e.g., 10% human serum, 1% GLUTAMAX™ in A-DMEM). The media used to culture the PCs may comprise an inhibitor of rho-associated coiled-coil containing protein kinase (ROCK), one example of which is Y27632.

Reprogramming factors may be used to reprogram the PCs such that they differentiate into iPSCs. In one aspect, obtaining iPSCs comprises transfecting PCs with RNA encoding reprogramming factors. The reprogramming factors may be octamer-binding transcription factor (Oct4) protein, Sry-box transcription factor 3 (Sox2) protein, Kruppel like factor 4 (Klf4) protein, cMyc protein, Nanog homeobox protein (Nanog), or Lin28 protein (Lin28). Thus, in one aspect, obtaining iPSCs comprises transfecting PCs with RNA encoding one or more reprogramming factors selected from the group consisting of octamer-binding transcription factor (Oct4) protein, Sry-box transcription factor 3 (Sox2) protein, Kruppel like factor 4 (Klf4) protein, cMyc protein, Nanog homeobox protein (Nanog), and Lin28 protein (Lin28). In one aspect, obtaining iPSCs comprises transfecting PCs with one or more RNA molecules encoding octamer-binding transcription factor (Oct4) protein, Sry-box transcription factor 3 (Sox2) protein, Kruppel like factor 4 (Klf4) protein, cMyc protein, Nanog homeobox protein (Nanog), and Lin28 protein (Lin28). Each reprogramming factor may be encoded by a separate RNA molecule. Alternatively, an RNA molecule may encode one or more reprogramming factor. In addition to the reprogramming factors disclosed above, the PCs may also be transfected with RNA molecules encoding one or more immune evasion proteins. Examples of useful immune evasion proteins are disclosed herein, and include, but are not limited to, vaccinia virus ubiquitin ligase (E3), vaccinia virus K3 protein, and vaccinia virus ankyrin repeat protein (B18). In one aspect, the PCs are further transfected with RNA molecules encoding one or more immune evasion proteins selected from the group consisting of vaccinia virus E3 protein, vaccinia virus K3 protein, and vaccinia virus protein B18 protein. In one aspect, the PCs are further transfected with RNA molecules encoding vaccinia virus E3 protein, vaccinia virus K3 protein, and vaccinia virus B18 protein. In one aspect, the PCS are further transfected with reprograming-enhancing, mature, micro-RNAs.

General methods of reprogramming PCs are known in the art. For example, one method comprises using a reprogramming kit such as the STEMGENT® StemRNA-NM Reprogramming kit available from REPROCELL, Inc. (Cat #00-0076) and related protocols described therein.

Transfected PCs may be cultured using appropriate medium designed to support growth and expansion of undifferentiated stem cells, human induced pluripotent stem cells (hiPSCs) and human mesenchymal stem cells (hMSC). Examples of such media include, but are not limited to, NutriStem hPSC X (STEMGENT; Cat. #01-0005) and mTesR™ 1 (STEMCELL TECHNOLOGIES; Cat. #05850).

Reprogramming of PCs using reprogramming factors disclosed herein results in iPSCs expressing pluripotent cell markers. In one aspect, the reprogrammed iPSCs express markers indicative of pluripotent stem cells. In one aspect, the markers comprise Oct3/4 proteins (encoded by the Pou5f1 gene) or stage-specific embryonic antigen 4 (SSEA-4) protein. In one aspect, reprogramming of PCs using reprogramming factors disclosed herein results in iPSCs expressing Oct3/4 and SSEA-4. In one aspect, the iPSCs have reduced expression, or lacked expression, of KRT5.

iPSCs may be used to produce iBCs by subjecting the iPSCs to a stepwise culture protocol in which the iPSCs differentiate into definitive endoderm (DE), which differentiate into anterior foregut endoderm (AFE), which differentiate into lung progenitor cells that can be used to produce iBCs. In one aspect, the iPSCs are first cultured under conditions such that the iPSCs differentiate into definitive endoderm cells (DECs). In one aspect, the iPSCs are cultured using 2D culture methodology. In one aspect, the culture conditions may comprise culturing the iPSCs on plates comprising at least one extracellular matrix protein, such as laminin. In one aspect, the iPSCs are cultured in medium designed to support growth and expansion of undifferentiated stem cells, human induced pluripotent stem cells (hiPSCs) and/or human mesenchymal stem cells (hMSC). Examples of such media include, but are not limited to, NutriStem hPSC X (STEMGENT; Cat. #01-0005) and mTesR™ 1 (STEMCELL TECHNOLOGIES; Cat. #05850). In one aspect, the medium may contain a ROCK inhibitor. Once established, the iPSCs may be fed using an appropriate medium, such as Medium 1 from STEMDIFF™ Definitive Endoderm Kit (STEMCELL TECHNOLOGIES; Cat. #05110). Medium 1 comprises MR and CJ supplements in Basal Medium. In certain aspects, after an appropriate period of time (e.g., 24 hours) in Medium 1, the cells may be fed using an appropriate medium, such as Medium 2 from STEMDIFF™ Definitive Endoderm Kit (STEMCELL TECHNOLOGIES; Cat. #05110). Medium 2 comprises CJ supplement in Basal Medium. Such culture conditions may be used to direct the iPSCs to differentiate into definitive endoderm cells (DECs).

In one aspect, the DECs do not express OCT3/4 protein or SOX2 protein (i.e., they are OCT3/4− and SOX2−). In one aspect, the DECs express CD184 (C-X-C chemokine receptor type 4(CXCR4)) and CD177 (tyrosine-protein kinase Kit protein (c-KIT)). In one aspect, the DECs are OCT3/4−, SOX2−, CD184+, and/or CD177+. In one aspect, the DECs are OCT3/4−, SOX2−, CD184+, and CD177+.

The DECs may be further cultured to form anterior foregut endoderm (AFE). Thus, in one aspect, the DECs are cultured under conditions such that they differentiate into AFE. In one aspect, the culture conditions may comprise culturing the DECs on plates comprising at least one extracellular matrix protein, such as laminin. In one aspect, the culture conditions comprise using 2D culture methodology. In one aspect, the culture conditions may comprise using an Anteriorization medium, which may comprise, or consist of, complete serum-free differentiation medium (CSFDM) comprising an inhibitor of a bone morphogenic protein (BMP) pathway (e.g., Dorsomorhpin) and a selective inhibitor of transforming growth factor beta (TGF-β)/Activin/NODAL/pathway (e.g., SB431542). In such culture conditions, the DECs differentiate into AFE. In one aspect, the AFE ells express forkhead box A2 (FOXA2) protein or SOX2 protein. In one aspect, the AFE ells express forkhead box A2 (FOXA2) protein and SOX2 protein. Following incubation in Anteriorization medium, the medium may be switched to Lung Progenitor Medium, which may comprise, or consist, of CSFDM comprising an inhibitor of glycogen synthase kinase-3 (GSK3) (e.g., CHIR99021), bone morphogenic protein 4 (BMP4), and retinoic acid. Under such conditions, the AFE further differentiate into Ventralized AFE containing lung progenitor cells. In one aspect, lung progenitor cells express NKX2.1 protein. In one aspect, lung progenitor cells are NKX2.1+. In one aspect, lung progenitor cells are FOXA2⁺, SOX2⁺, and NKX2.1⁺. In one aspect, a population comprising lung progenitor cells (i.e., FOXA2⁺, SOX2⁺, and NKX2.1⁺) may be enriched for lung progenitor cells (i.e., FOXA2⁺, SOX2⁺, and NKX2.1⁺ cells) using sorting techniques, such as anti-carboxypeptidase M(CPM) antibody-based live cell sorting.

Lung progenitor cells produced as described herein may be used to produce lung organoids, which may be used to produce iBCs. In one aspect, lung progenitor cells are optionally isolated and purified, and then cultured under conditions such that they form lung organoids. In one aspect, the culture conditions may comprise culturing the lung progenitor cells in a gelatin-based 3D organoid culture. In one aspect, the culture conditions may comprise culturing the lung progenitor cells on plates comprising a medium that mimics the environment of a basement membrane layer. The medium may be gelatinous. In one aspect, the culture conditions may comprise culturing the lung progenitor cells on plates comprising one or more extracellular matrix proteins, such as laminin, collagen IV, entactin, and heparan sulfate proteoglycan. One example of a medium suitable for culturing the lung progenitor cells is MATRIGEL®. Once the lung progenitor cells have incubated for a period of time, the culture medium may be supplemented with at least one of cAMP, a non-selective phosphodiesterase inhibitor (e.g., IBMX), basic fibroblast growth factor (bFGF), fibroblast growth factor 10 (FGF10) and dexamethasone. In one aspect, the medium is supplemented with cAMP, a non-selective phosphodiesterase inhibitor, bFGF, FGF10, and dexamethasone. In one aspect, a ROCK inhibitor is added to the medium. In one aspect, after about 1, about 2, or about 3 weeks, the lung organoid medium may be replaced with a medium designed for the expansion and differentiation of human airway cells, one example of which is PNEUMACULT™-ALI (STEMCELL TECHNOLOGIES; Cat. #05021). Incubation of such cultures will result in the formation of epithelial organoids, which may be used to produce iBCs.

To produce iBCs, the airway epithelial organoids may dissociated using appropriate means (e.g., mechanical disruption, enzymes such as Trypsin, Dispase, etc.), and the resulting epithelial cells cultured in the presence of fibroblast cells, which may be gamma-irradiated fibroblast cells. The fibroblasts may be in the form of a fibroblast feeder layer. In one aspect, the combined fibroblast and airway epithelial cell mixture may be cultured under conditions such that the epithelial cells differentiate into iBCs. In one aspect, the culture conditions comprise using a medium that supports the growth of a variety of mammalian cells, such as glial cells, fibroblasts, and endothelial cells (e.g., F-Medium), and optionally comprising one or more Duel-SMAD-inhibitors (e.g., DMH-1, A83-01, etc.), and a ROCK inhibitor (e.g., Y27632). Such conditions will result in a culture comprising iBCs, which are KRT5+, TP63+, and NKX2.1+. In one aspect, such conditions result in a population of cells that is at least 80%, at least 85%, at least 90%, at least 95%, or 100% triple-positive for KRT5/TP63/NKX2.1 (i.e., KRT5⁺, TP63⁺, NKX2.1⁺). In one aspect, the iBCs may be VIM-. The iBCs may be purified from the fibroblast/iBC cell mixture.

One embodiment is an induced basal cell (iBC) produced according to the methods disclosed herein. In one aspect, the iBC is an induced airway basal stem cell. In one embodiment the iBC is an induced airway epithelial basal cell. In one aspect, the iBC is an induced nasal basal cell or an induced bronchial basal cell. In one aspect, the iBC is a human iBC. In one aspect, the induced BC is KRT5⁺, TP63⁺, NKX2.1⁺.

One embodiment is a composition comprising a population of iBCs, wherein the population is at least 90%, at least 95%, at least 98% or 100%, KRT5⁺, TP63⁺, NKX2.1⁺. In one aspect, the iBCs are induced airway basal stem cells. In one aspect the iBCs are induced airway epithelial basal cells. In one aspect, the iBCs are induced nasal basal cells or induced bronchial basal cells. In one aspect, the iBCs are human iBCs.

One embodiment is a method of producing an epithelial tissue, comprising culturing one or more iBCs produced using a method disclosed herein, under conditions suitable for formation of an epithelial tissue. In one aspect, such conditions comprise culturing the one or more iBCs in an air-liquid interface culture. In one aspect, the one or more iBCs are induced airway basal stem cells. In one aspect, the one or more iBCs are induced airway epithelial basal cells. In one aspect, the one or more iBCs are induced nasal basal cells or induced bronchial basal cells. In one aspect, the one or more iBCs are human iBCs. In one aspect, the one or more iBCs comprise a population of iBCs that are at least 90%, at least 95%, at least 98% or 100%, KRT5⁺, TP63⁺, NKX2.1⁺.

One embodiment is an epithelial tissue comprising an iBC produced according to the disclosed methods. In one aspect, the epithelial tissue may be produced by culturing one or more iBCs, produced as disclosed herein, under conditions suitable for formation of an epithelial tissue. In one aspect, such conditions comprise culturing the one or more iBCs in an air-liquid interface culture. In one aspect, the one or more iBCs are induced airway basal stem cells. In one aspect, the one or more iBCs are induced airway epithelial basal cells. In one aspect, the one or more iBCs are induced nasal basal cells or induced bronchial basal cells. In one aspect, the one or more iBCs are human iBCs.

One embodiment is a method of treating an individual in need of such treatment, comprising administering to the individual an iBC, or an epithelial issue, produced according to the methods disclosed herein. In one aspect, the individual has a respiratory disease. In one aspect, the iBC or epithelial tissue is administered to treat the individual for a respiratory disease. In one aspect, administration of the iBC or epithelial tissue comprises transplanting the iBC or epithelial tissue into the subject's epithelium, which may comprise nasal epithelium, oral epithelium, pharyngeal epithelium, laryngeal epithelium, tracheal epithelium, bronchial epithelium, and/or lung epithelium. In one aspect, the iBC is an induced airway basal stem cell. In one embodiment the iBC is an induced airway epithelial basal cell. In one aspect, the iBC is an induced nasal basal cell or an induced bronchial basal cell. In one aspect, the iBC is a human iBC. In one aspect, the epithelial tissue comprises an induced airway basal stem cell. In one embodiment the epithelial tissue comprises an induced airway epithelial basal cell. In one aspect, the epithelial issue comprises an induced nasal basal cell or an induced bronchial basal cell. In one aspect, the epithelial tissue is a human epithelial tissue.

One embodiment is use of a method disclosed herein, in preparing an iBC. In one aspect, the iBC is an induced airway basal stem cell. In one embodiment the iBC is an induced airway epithelial basal cell. In one aspect, the iBC is an induced nasal basal cell or an induced bronchial basal cell. In one aspect, the iBC is a human iBC.

One embodiment is use of an iBC produced using a method disclosed herein, in preparing an epithelial tissue. Such use may comprise culturing one or more iBCs produced using a method disclosed herein, under conditions suitable for formation of an epithelial tissue. In one aspect, the iBC is an induced airway basal stem cell. In one embodiment the iBC is an induced airway epithelial basal cell. In one aspect, the iBC is an induced nasal basal cell or an induced bronchial basal cell. In one aspect, the iBC is a human iBC. In one aspect, the one or more iBCs comprise a population of iBCs that are at least 90%, at least 95%, at least 98% or 100%, KRT5⁺, TP63⁺, NKX2.1⁺.

One embodiment is use of an IBC or an epithelial tissue prepared using a method disclosed herein, in preparing a primary cell or tissue-based model of a disease. The use may comprise culturing an iBC, or an epithelial tissue, produced using a disclosed method, to prepare a model respiratory surface. In one aspect, the use may comprise administering an iBC, or an epithelial tissue, produced using a disclosed method, to an animal to produce an animal model that may be used to study a disease or its treatment. In one aspect, the primary cell or tissue-based model comprise an animal (e.g., mouse) to which an iBC or epithelial tissue produced using methods disclosed herein, has been administered. Such animals may be used to study a disease, particularly respiratory disease. For example, such animals may be used to test a response to a compound or environmental stimulus.

One embodiment is use of an IBC or an epithelial tissue prepared using a method disclosed herein, in studying a biological response to a compound or an environmental stimulus.

One embodiment is use of the disclosed methods, of an iBC or epithelial tissue of the disclosure, in the preparation of a medicament or therapeutic composition for treating a respiratory disease. In one aspect, the respiratory disease may be a chronic respiratory disease.

One embodiment is use of the disclosed methods, of an iBC or epithelial tissue of the disclosure, in identifying a therapeutic compound. The compound may be for the treatment of a respiratory disease. Such a use may comprise contacting a test compound with an iBC or epithelial tissue of the disclosure and measuring one or more characteristic of the iBC or epithelial tissue to identify changes thereto. Such changes may identify the test compound as a therapeutic compound. In one aspect, the compound is contacted with the iBC or epithelial tissue in an in vitro culture. In one aspect, the compound is contacted with the iBC or epithelial tissue in an animal to which the iBC or epithelial tissue has been administered.

One embodiment is a kit for practicing methods disclosed herein. A kit may comprise reagents and/or instructions for producing an iBC from an iPSC. A kit may also comprise reagents and/or instructions for using an iBC, or epithelial tissue, produced using a method of the disclosure, for treating a respiratory disease, examples of which include, but are not limited to, such as obstructive pulmonary disease, cystic fibrosis, and asthma. A kit may also comprise bottles, b buffers, tubes, syringes, needles, and the like.

The following experimental results are provided for purposes of illustration and are not intended to limit the scope of the invention.

### Examples

The following Tables provide examples of reagents and media, some of which are commonly available, suitable for practicing the disclosed methods. It should be understood that such media are provided as exemplary and that equivalent reagents or media providing equivalent, necessary functionality may be used.

TABLE 1
Reagents

| | | |
|---|---|---|
| REAGENT or RESOURCE | SOURCE | IDENTIFIER |

Antibodies

| | | |
|---|---|---|
| Chicken polyclonal anti-KRT5 (clone | BioLegend | Cat#905901; RRID: |
| Ploy9059) | | AB_2565054 |
| Mouse monoclonal anti-TP63 (clone 4A4) | Santa Cruz | Cat#sc-8431; RRID: |
| | Biotech | AB_628091 |
| Mouse monoclonal anti-CPM (clone WK) | FUJIFILM Wako | Cat#014-27501 |
| Mouse monoclonal anti-MUC5AC (clone | ThermoFisher | Cat#MA1-38223; RRID: |
| 45M1) | | AB_2146842 |
| Rabbit polyclonal anti-MUC5B (clone H- | Santa Cruz | Cat#sc-20119; RRID: |
| 300) | Biotech | AB_2282256 |
| Mouse monoclonal anti-Acetylated | Sigma-Aldrich | Cat#T6793; RRID: |
| Tubulin(clone6-11B-1) | | AB_477585 |
| Rabbit polyclonal anti-SCGB1A1 | BioVendor Lab | Cat# RD18022220; |
| | Med | RRID: AB_2335634 |
| Rabbit polyclonal anti-KRT8 | Atlas antibodies | Cat# HPA049866, RRID: |
| | | AB_2680923 |
| Rabbit monoclonal anti-NKX2.1 (clone | Abcam | Cat# AB227652 |
| SP141) | | |
| Mouse monoclonal anti-Vimentin (clone | Santa Cruz | Cat# sc-6260, RRID: |
| V9) | Biotech | AB_628437 |
| PE Mouse monoclonal anti-CXCR4 | STEMCELL | Cat#60089PE.1 |
| IgG2a (clone 12G5) | TECH | |
| APC Mouse monoclonal anti-c-Kit | Invitrogen | Cat#CD11705; RRID: |
| IgG1(clone 104D2) | | A_2536476 |
| Human Pluripotent Stem Cell Marker | R&D Systems | Cat# SC008 |
| Antibody Panel | | |
| Positive Markers | | |
| Mouse Anti-Human Alkaline | | |
| Phosphatase Monoclonal Antibody | | |
| Goat Anti-Human Nanog Antigen- | | |
| affinity Purified Polyclonal | | |
| Antibody | | |
| Goat Anti-Human Oct-3/4 | | |
| Antigen-affinity Purified | | |
| Polyclonal Antibody | | |
| Mouse Anti-Human SSEA-4 | | |
| Monoclonal Antibody | | |
| Negative Marker | | |
| Mouse Anti-Human SSEA-1 | | |
| Monoclonal Antibody | | |
| StainAlive TRA-1-60, Dylight 488, | Stemgent | Cat# 09-0068 |
| mouse anti-human | | |
| AlexaFluor 647 Donkey Anti-Mouse | Invitrogen | Cat# A32787; RRID: |
| IgG (H + L) | | AB_2762830 |
| AlexaFluor 647 Donkey Anti-Rabbit | Invitrogen | Cat#A-31573; |
| IgG (H + L) | | RRID: 2536183 |
| AlexaFluor 596 Donkey Anti-Mouse | Invitrogen | Cat#A-21203; RRID: |
| IgG(H + L) | | AB_2535789 |
| AlexaFluor 594 Donkey Anti-Rabbit | Invitrogen | Cat#A-21207; |
| IgG(H + L) | | RRID: 141637 |
| AlexaFluor 488 Donkey Anti-chicken | Jackson | Cat#703-545-155; RRID: |
| IgG(H + L) | ImmunoResearch | AB_2340375 |
| | Lab | |
| ProLong Diamond Antifade Mountant | Invitrogen | Cat# P36970 |

Biological Samples

| | | |
|---|---|---|
| Healthy Human Tracheal tissue | National Jewish | www.nationaljewish.org |
| | Health Tissue | |
| | Bank | |
| Healthy Human Nasal and bronchial | National Jewish | www.nationaljewish.org |
| brushing | Health Tissue | |
| | Bank | |

Chemicals, Peptides, and Recombinant Proteins

| | | |
|---|---|---|
| Growth Factor Reduced MATRIGEL ® | Corning | Cat# 356230 |
| Gelatin type A | Sigma-Aldrich | Cat#G1890-100G |
| Dispase | Corning | Cat#CB-40235 |
| Dorsomorphin | Stemgent | Cat#04-0024 |
| SB431542 | Tocris | Cat#1614 |
| CHIR99021 | Tocris | Cat#4423 |
| Retinoic acid | Sigma | Cat#R2625 |
| Y27632 dihydrochloride (ROCK | APExBio | Cat#A3008 |
| inhibitor) | | |
| (2S)-N-[(3,5-Difluorophenyl)acetyl]-L- | Selleck | Cat#S2215 |
| alanyl-2-phenyl]glycine 1,1-dimethylethyl | Chemicals | |
| ester (DAPT) | | |
| DMH-1 | ThermoFisher | Cat#41-261-0 |
| A83-01 | Sigma | Cat#SML0788 |
| DL-Dithiothreitol (DTT) | Sigma | Cat#D0632 |
| 8-bromoadenosine 30,50-cyclic | Sigma | Cat#B7880 |
| monophosphate | | |
| sodium salt (cAMP) | | |
| 3-Isobutyl-1-methylxanthine (IBMX) | Sigma | Cat#15879 |
| Recombinant human bFGF | STEMCELL | Cat#78003 |
| | TECH | |
| Recombinant human FGF10 | ThermoFisher | Cat#10573HNAE25 |
| Recombinant human BMP4 | ThermoFisher | Cat#PHC9534 |
| Dexamethasone | Sigma | Cat#D4902 |
| Protease from Strephtomyces griseus | Sigma | Cat#P5147 |
| 100X | Fisher | Cat#ICN1674049 |
| Penicillin/Strepotomycin/AmphtericinB | | |
| (PSA) | | |
| PFA | ThermoFisher | Cat#AA433689M |
| TritonX-100 | Fisher Scientific | Cat#9002-93-1 |
| DAPI | Sigma | Cat#D9542-5MG |
| HistoChoice | Sigma | Cat#H2779-1L |
| Antigen Unmasking Solution (Citric acid- | VECTOR LAB | Cat#H-3300 |
| based) | | |

Critical Commercial Assays

| | | |
|---|---|---|
| Quick-RNA MiniPrep Kit | Zymo Research | Cat#R1055 |
| Maxima First Strand cDNA Synthesis kit | ThermoFisher | Cat#K1642 |
| Brilliant III Ultra-fast qPCR master Mix | Agilent Tech | Cat#600880 |
| QuantStudio 6 Flex Real-Time PCR | Life Technologies | Cat#4485691 |
| System | | |
| ALS CellCelector platform | Automated Lab | Als-jena.com |
| | Solution | |
| Chromium Next GEM Single Cell 3′ Kit | 10xGenomics | Cat#1000268 |
| v3.1 | | |

Experimental Models: Cell Lines

| | | |
|---|---|---|
| Human healthy donor iPSC line (nasal) | Seibold Lab | |
| Human health donor iPSC line (bronchial) | Seibold Lab | |
| Human newborn donor fibroblast | ThermoFisher | Cat#GSC3404 |
| Mouse fibros for feeders | | |

Oligonucleotides

| | | |
|---|---|---|
| TaqMan Gene Expression Assay Primers | N/A | N/A |
| OCT4 | IDT | Hs.PT.58.14494169.g |
| SOX17 | IDT | Hs.PT.58.24876513 |
| FOXA2 | IDT | Hs.PT.58.26032236 |
| SOX2 | IDT | Hs.PT.58.237897.g |
| NKX2.1 | IDT | Hs.PT.58.2461055 |
| TP63 | IDT | Hs.PT.58.2966111 |
| KRT5 | IDT | Hs.PT.51.1920889.gs |
| GUS B | IDT | Hs.PT.51.2648420 |

Software and Algorithms

| | | |
|---|---|---|
| Affinity Designer | Graphics Editor | https://affinity.serif.com |
| ImageJ | NIH | https://imagej.nih.gov/ij/ |
| FlowJo | FlowJo, LLC | www.flowjo.com |
| Cell Ranger | 10X Genomics | www.10xgenomics.com |

Other

| | | |
|---|---|---|
| STEMDIFF ™Definitive Endoderm Kit | STEMCELL | Cat#05110 |
| STEMDIFF ™Endoderm Basal | TECH | |
| Medium | | |
| STEMDIFF ™Definitive | | |
| Endoderm Supplement MR | | |
| STEMDIFF ™Definitive | | |
| Endoderm Supplement CJ | | |
| mTeSR1 | STEMCELL | Cat#05850 |
| | TECH | |
| iMatrix 511 | REPROCELL | Cat#NP892-011 |
| Gentle Cell Dissociation Reagent | STEMCELL | Cat#07174 |
| | TECH | |
| 6.5 mm Transwell with 0.4 mm Pore | Corning | Cat#3470 |
| Polyester | | |
| Membrane Inserts, Sterile | | |
| PNEUMACULT ™-ALI Medium | STEMCELL | Cat#05021 |
| | TECH | |
| PNEUMACULT ™-Ex Plus Medium | STEMCELL | Cat#05040 |
| | TECH | |
| ES Qualified FBS | Thermo Fisher | Cat#16141 |
| ES-DMEM | Thermo Fisher | Cat#GSM-2001 |
| A-DMEM | ThermoFisher Sci | Cat#12491015 |
| Human Serum | Sigma | Cat#H4522 |
| GlutaMax Supplement | ThermoFisher Sci | Cat#35050061 |
| 0.05% Trypsin/EDTA | ThermoFisher Sci | Cat#25300054 |
| AggreWell 400 24-well Plate | STEMCELL | Cat#34411 |
| | TECH | |
| AggreWell Rinsing Solution | STEMCELL | Cat#07010 |
| | TECH | |
| CryoStem freezing media | Stemgent | Cat#01-0013-50 |
| Bacillus licheniformis protease | Sigma | Cat#P5380 |
| Deoxyribonuclease I (DNase I) | Sigma | Cat#DN25 |
| Ethylenediaminetetraacetic acid (EDTA) | ThermoFisher Sci | Cat#BP2482 |
| STEMDIFF ™Trilineage Differentiation | STEMCELL | Cat#05230 |
| Kit | TECH | |
| StemRNA-NM Reprogramming kit | REPROCELL | Cat#00-0076 |
| 3-Germ Layer Immunocytochemistry Kit | ThermoFisher Sci | Cat# A25538 |
| (Anti-TUJ1, anti-AFP, and anti-SMA | | |
| primary antibodies) | | |
| Human Pluripotent Stem Cell Trilineage | STEMCELL | Cat#07515 |
| Differentiation qPCR Array | TECH | |
| Dynabeads Pan Mouse IgG | Invitrogen | Cat#11041 |
| NutriStem XF/FF culture media | Stemgent | Cat#01-0005 |
| DMEM-F | Fisher | Cat#SH3024301 |
| Cell strainer, 40 um | Fisher | Cat#08-771-2 |
| Knockout Serum Replacement medium | ThermoFisher | Cat#10828010 |
| Fixation/Permeabilization Solution Kit | BD biosciences | Cat#554714 |
| Flow Cytometry Buffer | R&D systems | Cat#FC001 |
| Nanodrop | ThermoFisher | Cat#ND-1000 |

TABLE 2
A-DMEM

| Component | Concentration (mg/L) |
|---|---|
| Glycine | 37.5 |
| L-Alanine | 8.9 |
| L-Arginine hydrochloride | 84.0 |
| L-Asparagine | 13.2 |
| L-Aspartic acid | 13.3 |
| L-Cystine 2HCl | 63.0 |
| L-Glutamic Acid | 14.7 |
| L-Histidine hydrochloride-H2O | 42.0 |
| L-Isoleucine | 105.0 |
| L-Leucine | 105.0 |
| L-Lysine hydrochloride | 146.0 |
| L-Methionine | 30.0 |
| L-Phenylalanine | 66.0 |
| L-Proline | 11.5 |
| L-Serine | 52.5 |
| L-Threonine | 95.0 |
| L-Tryptophan | 16.0 |
| L-Tyrosine disodium salt dihydrate | 104.0 |
| L-Valine | 94.0 |
| Ascorbic Acid phosphate | 2.5 |
| Choline chloride | 4.0 |
| D-Calcium pantothenate | 4.0 |
| Folic Acid | 4.0 |
| Niacinamide | 4.0 |
| Pyridoxine hydrochloride | 4.0 |
| Riboflavin | 0.4 |
| Thiamine hydrochloride | 4.0 |
| i-Inositol | 7.2 |
| Calcium Chloride (CaCl2) (anhyd.) | 200.0 |
| Ferric Nitrate (Fe(NO3)3″9H2O) | 0.1 |
| Magnesium Sulfate (MgSO4) (anhyd.) | 97.67 |
| Potassium Chloride (KCl) | 400.0 |
| Sodium Bicarbonate (NaHCO3) | 3700.0 |
| Sodium Chloride (NaCl) | 6400.0 |
| Sodium Phosphate dibasic (Na2HPO4—H2O) | 125.0 |
| AlbuMAX ® II | 400.0 |
| Human Transferrin (Holo) | 7.5 |
| Insulin Recombinant Full Chain | 10.0 |
| Ammonium Metavanadate | 3.0E−4 |
| Cupric Sulfate | 0.00125 |
| Manganous Chloride | 5.0E−5 |
| Sodium Selenite | 0.005 |
| D-Glucose (Dextrose) | 4500.0 |
| Ethanolamine | 1.9 |
| Glutathione (reduced) | 1.0 |
| Phenol Red | 15.0 |
| Sodium Pyruvate | 110.0 |

TABLE 3
IMDM

| Component | mg/L |
|---|---|
| Calcium Chloride Anhydrous | 165 |
| Dextrose | 4.5E+03 |
| Magnesium Sulfate Anhydrous | 97.66 |
| Potassium Chloride | 330 |
| Sodium Bicarbonate | 3.024E+03 |
| Sodium Chloride | 4.505E+03 |
| Sodium Selenite (Platelet factor enriched serum) | 1.130E−02 |
| L-alanine | 25 |
| L-arginine monochloride | 84 |
| L-asparagine | 24.989 |
| L-aspartic acid | 30 |
| L-glutamic acid | 75 |
| L-glutamine | 584 |
| Glycine | 30 |
| L-histidine monochloride, anhydrous | 42 |
| L-isoleucine | 104.8 |
| L-leucine | 104.8 |
| L-Lysine monohydrochloride | 146.2 |
| L-methionine | 30 |
| L-phenylalanine | 60 |
| L-proline | 40 |
| L-serine | 42 |
| L-threonine | 95.2 |
| L-tryptophan | 16 |
| L-valine | 93.6 |
| D-biotin (vitamin H) | 1.3E−02 |
| D-Calcium Pantothenate (Vitamin B5) | 4 |
| Choline chloride | 4 |
| Cyanocobalamin (Vitamin B12) | 1.3−02 |
| Folic acid | 4 |
| I-inositol | 7 |
| Niacinamide (nicotinamide) | 4 |
| Pyridoxine Monohydrochloride | 4 |
| Riboflavin (Vitamin B2) | .4 |
| Thiamine Monohydrochloride (Vitamin B1) | 4 |
| HEPES Buffer | 5.98E+03 |
| Phenol Red | 15.34 |
| Pyruvic Acid Sodium Salt | 110 |
| Potassium Nitrate | 0.076 |
| L-Tyrosine Disodium Salt, Dihydrate | 103.79 |
| L-Cystine Dihydrochloride | 91.24 |
| Sodium Phosphate monobasic, anhydrous | 108.69 |

TABLE 4
HAM'S F-12

| Component | g/L |
|---|---|
| Calcium chloride (Anhydrous) | 33.22 |
| Cupric sulfate (CuSO₄—5H₂O) | 0.0025 |
| Ferric sulfate (FeSO₄—7H₂O) | 0.834 |
| Potassium chloride (KCl) | 223.60 |
| Magnesium chloride (Anhydrous) | 57.22 |
| Sodium chloride (NaCl) | 7599.00 |
| Sodium bicarbonate (NaHCO₃) | 1176.00 |
| Sodium phosphate, dibas (Anhydrous) | 142.04 |
| Zinc sulfate (ZnSO₄—7H₂O) | 0.863 |
| D-Glucose | 1802.00 |
| Hypoxanthine Na | 4.77 |
| Linoleic Acid | 0.084 |
| Lipoic Acid | 0.21 |
| Phenol red | 1.20 |
| Putrescine-2HCl | 0.161 |
| Sodium Pyruvate | 110.00 |
| Thymidine | 0.73 |
| L-Alanine | 8.90 |
| L-Arginine hydrochloride | 211.00 |
| L-Asparagine-H₂O | 15.00 |
| L-Aspartic acid | 13.30 |
| L-Cysteine-HCl—H₂O | 35.12 |
| L-Glutamic acid | 14.70 |
| L-Glutamine | 146.00 |
| Glycine | 7.50 |
| L-Histidine-HCl—H₂O | 20.96 |
| L-Isoleucine | 3.94 |
| L-Leucine | 13.10 |
| L-Lysine hydrochloride | 36.50 |
| L-Methionine | 4.48 |
| L-Phenylalanine | 4.96 |
| L-Proline | 34.50 |
| L-Serine | 10.50 |
| L-Threonine | 11.90 |
| L-Tryptophan | 2.04 |
| L-Tyrosine | 5.40 |
| L-Valine | 11.70 |
| Biotin | 0.0073 |
| D-Calcium pantothenate | 0.48 |
| Choline chloride | 13.96 |
| Folic acid | 1.30 |
| i-Inositol | 18.00 |
| Niacinamide | 0.037 |
| Pyridoxine hydrochloride | 0.062 |
| Riboflavin | 0.038 |
| Thiamine hydrochloride | 0.34 |
| Vitamin B12 | 1.36 |
| D-glucose | 1.802 |
| Hypothanxine | 0.00408 |
| Linoleic acid | 0.000084 |
| Phenol Red | 0.0013 |
| Putrescine | 0.000161 |
| Pyruvic acid | 0.11 |
| Thioctic acid | 0.00021 |
| Thymidine | 0.00073 |
| L-glutamine | 0.146 |

TABLE 5
Fibroblast Expansion Medium

10% Human serum
1% GLUTAMAX ™ in A-DMEM

TABLE 6
Complete Serum-free Differentiation Medium (CSFDM)

| Component | Amount |
|---|---|
| IMDM | 75% |
| Ham's F12 | 25% |
| Ascorbic Acid | 50 μg/ml |
| B27 Supplement (ThermoFisher Scientific; | 0.5X |
| Cat. #17504-044) | |
| N2 Supplement (ThermoFisher Scientific; | 0.5X |
| Cat. #1750-2048) | |
| Bovine Serum Albumin | 0.05% |
| GLUTAMAX ™ | 1X |
| Monothioglycerol | 2 ng/ml |
| PRIMOCIN ™ (InvivoGen) | 100 μg/ml |

TABLE 7
Anteriorization Medium

| | |
|---|---|
| CSFDM | |
| Dorsomorphin (STEMGENT; Cat. #04-0024) | 2 μM |
| SB431542 (Tocris; Cat. #1614) | 10 μM |

TABLE 8
Lung Progenitor medium

| Component | Amount | |
|---|---|---|
| CSFDM | | |
| CHIR99021 (Tocris; Cat. #4423) | 3 | μM |
| BMP4 | 10 | ng/ml |
| Retinoic Acid | 100 | nM |

TABLE 9
Lung Organoid Expansion Medium

| Component | Amount | |
|---|---|---|
| cAMP | 100 | μM |
| 3-Isobutyl-1-methylxanthine (IBMX) | 100 | μM |
| (SIGMA; Cat. #15879) | | |
| Recombinant human basic fibroblast | 100 | ng/ml |
| growth factor | | |
| dexamethasone | 50 | nM |

TABLE 10
F-Medium

| Component | Amount | |
|---|---|---|
| DMEM-F | 67.5% | |
| Ham's F-12 | 25% | |
| Fetal Bovine Serum (FBS) | 7.5% | |
| L-Glutamine | 1.5 | mM |
| hydrocortisone | 25 | ng/ml |
| Epidermal growth factor (EGF) | 12.5 | ng/ml |
| Cholera Toxin | 8.6 | ng/ml |
| Adenine | 24 | ug/ml |
| insulin | 0.1% | |
| Pen/strep (optional) | 75 | U/ml |

### General Methods

The following section provides examples of methods suitable for generating, maintaining, manipulating, and analyzing cells of the disclosure. Such disclosure is not intended to limit the invention to any specific method but is considered exemplary. It should be understood related methods and minor modifications of the disclosed methods may also be used to produce and use cells of the disclosure.

All incubations were performed at 37° C., 5% CO₂, ambient O₂.

A. Human Trachea Sample

Human tracheal airway epithelium was isolated from a de-identified donor whose lungs were not suitable for transplantation. The specimen was obtained from the Donor Alliance of Colorado, and informed consent was obtained from authorized family members of the donor. The National Jewish Health Institutional Review Board (IRB) approved the research under IRB protocols HS-3209 and HS-2240.

### Tracheal Digest

The human trachea sample was wet in Stock solution (DMEM-F+1×PSA) and fat and connective tissue were removed, before cutting into small sections. Sections were rinsed in Stock solution to remove mucus before proteolytic digest (0.2% Protease in Stock solution) overnight at 4° C., with rocking. Protease was neutralized with FBS, the supernatant was saved (tube 1), and tracheal sections washed (5 mM HEPES, 5 mM EDTA, 150 mM NaCl) for 20 min at 37° C. The supernatant was also saved (tube 2) and the loosened epithelium was then manually scraped off into stock solution with 10% FBS (tube 3), and all cells were collected by centrifugation for 10 min at 225×g, 4° C. (tubes 1, 2, and 3). Cell pellets resuspended in BEGM+0.5×PSA were filtered using a 70μm cell strainer, collected by centrifugation (5 min, 225×g, 4° C.) and cryopreserved in freeze media (F-media, 30% FBS, 10% DMSO).

On the day of capture, cryopreserved CAP digestions (in vitro samples) or tracheal digest (in vivo sample) were quick thawed, washed twice in 1×PBS/BSA (0.04%) and resuspended at 1200 cells/uL for capture on the 10× Genomics platform.

B. Generation of Human, Induced, Pluripotent Stem Cells (hiPSCs)

Primary airway epithelial cells (human nasal and bronchial airway epithelial cells) and fibroblasts (human newborn foreskin fibroblasts) were reprogramed using the STEMRNA™-NM Reprogramming kit (REPROCELL; Cat. #00-0076), according to the manufacturer's protocol with initial numbers of cells seeded as listed in Table 1. Reprograming factors included a cocktail of synthetic factors (Oct4, Sox2, Klf4, cMyc, Nanog, and Lin28) and immune evasion factors (E3, K3, and B18), with reprogramming-enhancing mature, double-stranded microRNAs. Briefly, on Day 0, primary airway epithelial cells or fibroblasts were plated on iMatrix™-511 pre-coated tissue culture plates (REPROCELL; Cat. #NP892-011) in 2 mL of PNEUMACULT™-Ex Plus Medium (STEMCELL TECHNOLOGIES; Cat. #05040) (with 10 uM Rock Inhibitor) or Fibroblast Expansion Medium (10% human serum, 1% GLUTAMAX™ in A-DMEM), respectively. Starting on the following day, culture media was exchanged for NUTRISTEM® hPSC XF STEMGENT; Cat. #01-0005). Reprogramming factors were transfected in Opti-MEM/RNAIMAX® on Days 1˜4 according to the manufacturer's protocol, including 6⁺ hr rest in fresh NUTRISTEM® medium before subsequent transfections on days 2-4. From Day 5 onward, NUTRISTEM® medium was exchanged daily. From Day 10-19 for AECs and Day 20⁺ for fibroblasts, emerging iPSC colonies were identified, and intact OCT3/4+ colonies were picked by hand scraping or with a Cellcelector automated system into iMatrix™-coated 12-well or 24-well plates. Medium was exchanged for mTeSR1™ the following day, and mTeSR1™ was utilized for all subsequent daily media changes.

C. Maintenance of hiPSCs

Prior to differentiation, reprogramed human iPSCs were cultured as a monolayer on feeder-free iMatrix™-511-coated tissue culture plates in mTeSR1™ medium supplemented with ROCK inhibitor (10 uM). The cells were passaged every 3-5 days with 0.5 mM EDTA/1×PBS and sub-cultured at least 10 times to stabilize in reprogramed states.

D. In-Vitro Trilineage Differentiation of hiPSCs

To validate the pluripotency, iPSCs were differentiated to the three germ layers (ectoderm, mesoderm, and endoderm) using both targeted and spontaneous differentiation protocols.

Targeted differentiation: in vitro directed differentiation was performed with the STEMDIFF™ Trilineage differentiation kit (STEMCELL TECHNOLOGIES; Cat. #05230) according to the manufacturer's instructions. Briefly, cells were plated in mTeSR11™ (with 10 uM Rock Inhibitor) on iMatrix-511-coated 6-well tissue culture plates and incubated overnight. Next day, the medium was replaced with STEMDIFF™ Trilineage Ectoderm Medium, Mesoderm Medium, and Endoderm Medium. Differentiated cells were harvested and analyzed lineage-specific markers (Ectoderm: TFAP2A and DMRT3; Mesoderm: MSX1 and T; Endoderm: SOX17 and FOXA2) on day 5 (mesoderm and endoderm lineages) and day 7 (ectoderm lineage).
Spontaneous differentiation: AGGREWELL™ 400 Plates were used to generate embryoid bodies (EBs) in mTeSR1™ medium. After formation of intact EBs (24-48 hrs), EBs were carefully transferred to AGGREWELL™ 1 Rising Solution treated low-attachment 6-well tissue culture plates and differentiated 7 days in DMEM/F12 supplemented with 20% KNOCKOUT™ Serum Replacement medium (ThermoFisher; Cat. #10828010). Primary differentiated EBs were transferred to 0.1% Gelatin coated plates and further differentiated for 7 days in DMEM supplemented with 10% FBS. Differentiation of three germ layers was assessed by immunostaining with the 3-Germ Layer Immunocytochemistry Kit (ThermoFisher; Cat. #A25538) according to the manufacturer's protocol.

E. Directed Differentiation of iPSCs into Lung Progenitor Cells

Differentiation of iPSCs into definitive endoderm: Nasal, bronchial, and fibroblast-derived iPSCs were differentiated into definitive endoderm using the STEMDIFF™ Definitive Endoderm Kit (STEMCELL TECHNOLOGIES; Cat. #05110) according to the manufacture's protocol. Briefly, on day 0, single cells were plated onto MATRIGEL® (Corning; Cat. #356230) coated plates at a density of 2×10⁵ cells/cm² in mTeSR1™ medium supplemented with Rock inhibitor and incubated for 24 hrs. The next day (Day 1), cells were fed with Medium 1 (supplement MR and CJ in Basal Medium). From Day 2-4, cells were fed every day with Medium 2 (supplement CJ only in Basal Medium). On Day 5, cells were ready to be assayed for the definitive endoderm. The purity of definitive endoderm cells was assessed by flow cytometry after labeling with fluorochrome-conjugated anti-CXCR4 and anti-cKit antibodies.

Differentiation of definitive endoderm into anterior foregut and lung progenitor: After formation of definitive endoderm, cells were dissociated with Gentle Cell Dissociation Reagent (STEMCELL TECHNOLOGIES; Cat. #07174) for 3 mins at room temperature and passaged at a ratio 1:6 onto MATRIGEL®-coated tissue culture plates in Anteriorization Medium [Complete serum-free differentiation medium (CSFDM) supplemented with 2 uM Dorsomorphin and 10 uM SB431542]. CSFDM was composed of 75% IMDM, 25% Ham's F12, 50 μg/ml Ascorbic acid, 0.5×B27 supplement, 0.5×N2 supplement, 0.05% bovine serum albumin, 1× GLUTAMAX™, 2 ng/ml Monothioglycerol, and 100 m/ml Primocin. After 3 days anteriorization, the medium was switched to Lung Progenitor Medium (CSFDM supplemented with 3 uM CHIR99021, 10 ng/ml BMP4, and 100 nM Retinoic Acid) for 6 more days. On day 15 of differentiation, cells were dissociated with 0.05% Trypsin and resuspended in Flow cytometry buffer. The lung progenitors were enriched by anti-carboxypeptidase M (CPM) (FUJIFILM; Cat. #014-27501) antibody-based live cell sorting.

F. Differentiation of Lung Progenitors into Airway Epithelial Organoids

Purified lung progenitors were resuspended in undiluted MATRIGEL® at the concentration of 1,000 cells/ul and plated as 50 ul droplets in each well of 24-well tissue culture plate. After the drops were fully polymerized at 37 C°, Lung Organoids Expansion Medium [100 uM cAMP, 100 uM IBMX, 250 ng/ml bFGF, 100 ng/ml FGF10, and 50 nM Dexamethasone] was added, supplemented with 10 uM Rock Inhibitor for the first 24 hrs. Culture medium was changed every other day and cells started to form intact epithelial spheroids with clear lumen after a week or so. After 2 weeks, the lung organoid expanding medium was replaced with Lung Organoids Differentiation Medium (PNEUMACULT™-ALI) (STEMCELL TECHNOLOGIES; Cat. #05021) supplemented with 5 uM DAPT (Selleck Chemicals; Cat. #S2215) to induce airway epithelial differentiation for 2 more weeks.

G. Generation of Induced Airway Basal Cells from Epithelial Organoids

Differentiated airway epithelial organoids were dissociated and transferred onto gamma-irradiated fibroblast feeder layers. Briefly, Lung Organoids Differentiation Medium was carefully aspirated from each well and 2 U/ml dispase added to dissolve MATRIGEL® components at 37 C° for 1 hr. During incubation, pipetting with a 5 ml serological pipette helped dissociation. Dissociated organoids were collected and further treated with 0.25% Trypsin for 5 min, 37 C to make single cell suspensions. The trypsin was neutralized by adding 1% Fetal Bovine Serum. Finally, 5×10⁵ cells were plated onto a 10 cm dish containing a fibroblast feeder layer (seeded 1-3 days prior). Cells were fed with F-medium (67.5% DMEM-F, 25% Ham's F-12, 7.5% FBS, 1.5 mM L-glutamine, 25 ng/mL hydrocortisone, 12 5 ng/mL EGF, 8.6 ng/mL cholera toxin, 24 ug/mL Adenine, 0.1% insulin, 75 U/mL pen/strep) supplemented with Dual-SMAD inhibitors (1 uM each DMH-1 and A83-01) and Rock inhibitor (10 uM). Culture medium was changed every other day, with cells starting to form tight cell colonies after 3 days or so. iBCs were purified from the fibroblast feeder layer using a 2-step trypsinization protocol, where the first step encompassed treatment with 0.25% Trypsin at 37° C. for 1 min to remove the fibroblast fraction and the second step entailed 5 min of 0.25% Trypsin at 37° C. to detach the more adherent, tight basal cell colonies. iBCs may be passaged at least up to 8 times in the same manner.

H. Differentiation of BC and iBCs Via ALI Culture

1×10⁵ BCs or iBCs were seeded onto 1:100 diluted MATRIGEL®-coated 24-well transwell inserts in ALI expansion medium (PNEUMACULT™-Ex Plus) with Rock inhibitor. After 24 hrs, the cells were fed with ALI expansion medium without Rock inhibitor, both apically and basolaterally. After another 24 hrs, apical medium was removed and basolateral medium was replaced with ALI differentiation medium (PNEUMACULT™-ALI). Basolateral medium was exchanged for fresh ALI differentiation medium every 48 hrs for the subsequent 21 days.

I. Flow Cytometric Analysis and Flow Activated Cell Sorting

Characterization of iPSC's pluripotency marker expression were performed by flow cytometric analysis. 1×10⁶ Cells were fixed and permeabilized with CYTOFIX/CYTOPERM™ solution for 20 min on ice and washed twice with 1× Perm/Wash buffer. Cells were then incubated with primary antibodies for 30 min on ice followed by Alexa Fluor-conjugated secondary antibodies for 30 min on ice in the dark. Cells were washed twice and resuspended in an appropriate volume of Flow Cytometry Staining Buffer and Flow cytometric analysis performed (BD LSRII). Data were exported and analyzed by FlowJo software. For fluorescence activated cell sorting, live cells were incubated with anti-CPM primary antibody for 30 min on ice followed by Alexa Fluor-conjugated secondary antibody for 30 min on ice in the dark. Cells were washed twice and resuspended in an appropriate volume of Flow Sorting Buffer (10% FBS in PBS with Rock Inhibitor) and live cell sorting performed (BD Aria Fusion).

J. Immunocytochemistry Staining

Cytospins were used to immobilize cells onto glass microscope slides. Live cells were fixed in 4% paraformaldehyde (PFA) for 20 min at room temperature then permeabilized with 0.4% Triton X-100 for 10 min at room temperature. Cells were incubated with primary antibodies KRT5(1:5,000), KRT8(1:5,000), TP63(1:200), NKX2.1(1:200), and VIM(1:1,000) for 1 hr at room temperature followed by Alexa Fluor-conjugated secondary antibodies(1:1,000) for 45 min in the dark. For nuclear staining, cells were incubated with DAPI for 5 minutes. All stained cells were mounted with ProLong Diamond Mount Medium and imaged using an Echo Revolve R4 fluorescence microscope. For quantification of staining, 5 random 20× objective fields per slide were captured and counted with ImageJ software. For quantification of cell size, ImageJ software was used and followed by Baviskar's Method (The American Biology Teacher (2011)73(9):554-556).

K. Immunohistochemistry Staining

Human trachea tissues were fixed in 10% neutral buffered formalin and ALI cultures were fixed in 4% PFA. Tissues and ALI cultures were paraffin-embedded and sectioned onto microscope slides. Deparaffinization was performed with HistoChoice, followed by a standard Ethanol dilution series (100%, 90%, 70%, 50%, and 30%), and antigen retrieval in Antigen Unmasking before blocking in Blocking Buffer (1×PBS, 3% BSA, 0.1% TritonX-100). Histology sections were incubated with primary antibodies KRT5(1:5,000), TP63(1:200), MUC5AC(1:500), MUC5B(1:500), ACT(1:5,000), and SCGB1A1 (1:1000) for 1 hr at room temperature followed by Alexa Fluorochrome-conjugated secondary antibodies(1:1,000) for 45 min in the dark. For nuclear staining, cells were incubated with DAPI for 5 minutes. All stained tissues were mounted with ProLong Diamond Mount Medium and imaged using Echo Revolve R4 fluorescence microscope. For quantification of staining, 5 random 20× objective fields per slide were captured and the percentage of the total image area threshold was analyzed using Image) software. Sections were stained with DAPI to identify cell nuclei and were used to determine the area of the section.

L. RNA Isolation and Quantitative Real-Time PCR (qRT-PCR)

Total RNAs were extracted with the Quick-RNA MiniPrep Kit according to the manufacturer's protocol. Purity and concentration of RNA samples were assessed with NanoDrop ND-1000 Spectrophotometer. Reverse transcription was conducted with the Maxima First Strand cDNA Synthesis kit. Finally, qRT-PCR was performed with Brilliant III Ultra-fast qPCR master Mix plus 5′ PrimeTime TaqMan Assay on QuantStudio 6 Flex Real-Time PCR system. mRNA expression levels, relative to the GUSB housekeeping gene, were determined by the ddCT method.

M. Single-Cell RNA Sequencing

### Organoids

To make single cell suspension from organoids, carefully aspirated organoid differentiation medium and added 2 U/ml dispase to cover the organoids and incubated at 37° C. for 1 hr until MATRTGEL® is fully dissolved. Using a 1,000 ul wide orifice pipette tip transferred the dissociated organoids into a new 15 ml Conical tube and added an equal volume of DMEM. The intact organoids were collected by centrifugation for 5 min at 300×g, 4° C. Carefully aspirated the supernatant and added 1 ml of 0.25% Trypsin per dissociated drop and incubated for 10 min at 37 C. Collected single cell suspension was added to equivalent volume of stop medium (10% FBS/DMEM) and centrifuged for 5 min at 300×g, 4 C. The cell pellet was washed with cold PBS once. The final cell pellet was resuspended in PBS with 0.04% BSA for single-cell gene expression profiling with the 10× Genomics system.

N. iBCs

To purify iBCs from the fibroblast feeder layer, removed fibroblasts fraction first by treatment with 0.25% Trypsin at 37 C for 1 min and then detached the tight iBC colonies by treatment with 0.25% Trypsin at 37 C for 5 min. Collected single cell suspension from dishes were transferred to 15 ml Conical tube containing 1 ml of cold FBS. The cell pellet was washed with cold PBS once. The final cell pellet was resuspended in PBS with 0.04% BSA for single-cell gene expression profiling with the 10× Genomics system.

O. ALIs and iALIs

To collect cells from ALIs/iALIs, apical culture chambers were washed once with warm PBS and then with warm PBS supplemented with 10 mM DTT, followed by two PBS washes to remove residual DTT. Cold active protease (CAP) solution (2.5 ug/ml Bacillus licheniformis protease, 125 U/ml DNase, and 0.5 mM EDTA in DPBS w/o Ca2+Mg2+) was added to the apical culture chamber and incubated on ice for 10 min with mixing every 2.5 min. Dissociated cells in CAP solution were added to 500 μl cold FBS, brought up to 5 ml with cold PBS, and centrifuged at 225×g and 4° C. for 5 min. The cell pellet was resuspended in 1 mL cold PBS+ DTT, centrifuged at 225×g and 4° C. for 5 min, and then washed twice with cold PBS. The final cell pellet was resuspended in PBS with 0.04% BSA for single-cell gene expression profiling with the 10× Genomics system.

### Example 1

This example describes reprograming of primary airway epithelial cells (AECs) to iPSCs.

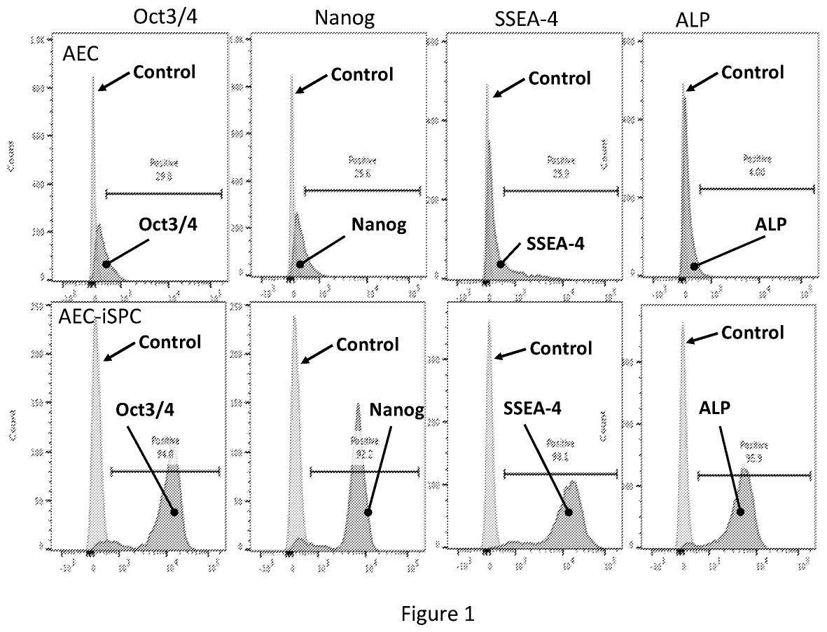

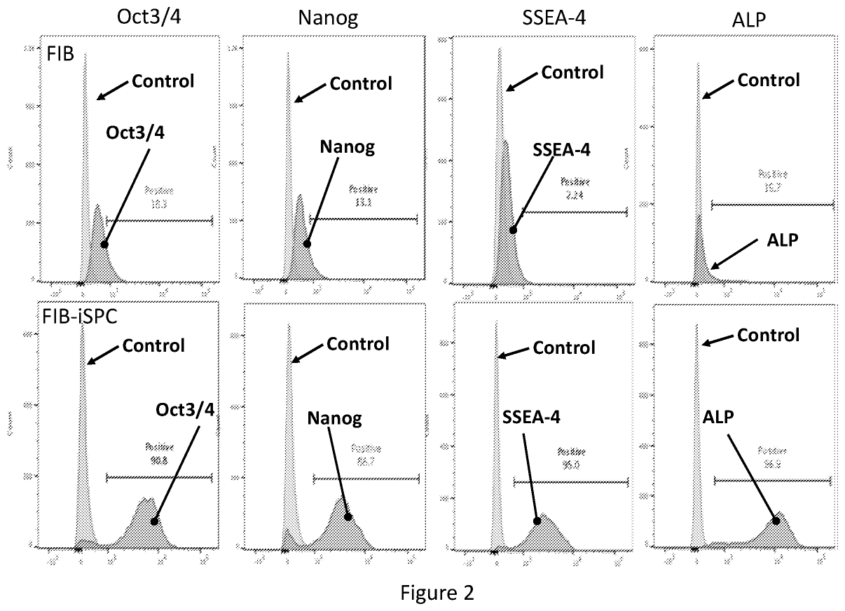

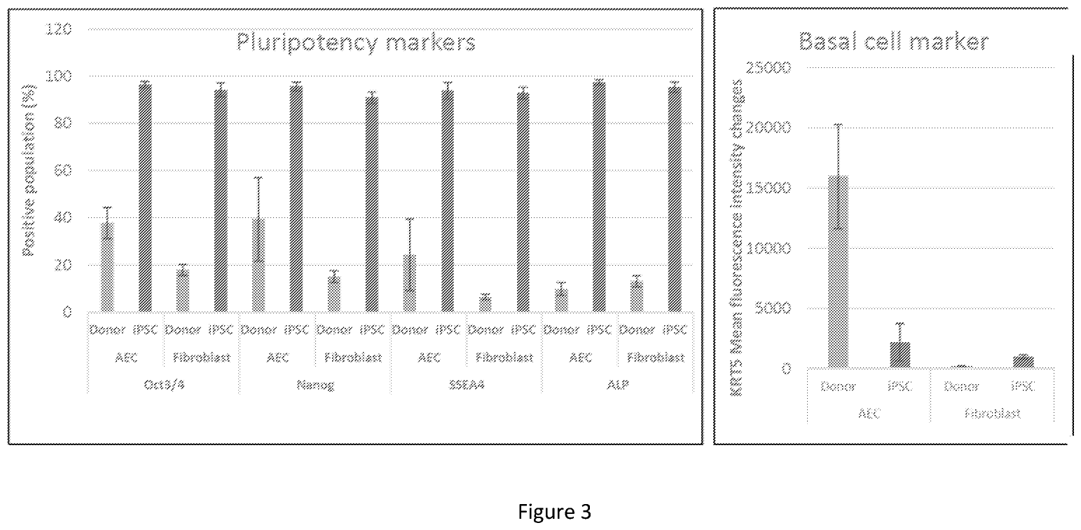

Expanded upper airway brushing AECs were transfected with a synthetic non-modified RNA cocktail consisting of reprogramming factors (Oct4, Sox2, Klf4, cMyc, Nanog, and Lin28), immune evasion factors (E3, K3, and B18) and reprogramming-enhancing mature microRNAs using a STEMDIFF™ RNA-NM Reprogramming kit. iPSC colonies were generated as early as 10 days post-transfection with these reprogramming factors, when individual colonies displayed abundant Oct3/4 and SSEA-4 by immunofluorescence (IF) labeling, which was absent in the surrounding un-reprogrammed cells. Isolated clones retained pluripotency markers and robust replicative capacity for at least 30 passages (FIGS. 1-3), similar to fibroblast-derived iPSCs following the same protocol (FIGS. 2 and 3).

Brushing AECs from 7 different donors were reprogrammed, including 2 from bronchial AECs and 5 from nasal AEC donors with reprograming efficiencies up to 0.16%. Karyotyping indicated that no chromosomal abnormalities were incurred during reprograming. To demonstrate these AEC-derived iPSC's pluripotent potential, three-germ layer differentiation was performed in vitro under both targeted and spontaneous differentiation conditions. RT-qPCR for germ layer specific markers confirmed distinct morphologies observed by targeted differentiation of iPSCs, where each germ layer exclusively expressed the expected markers regardless of the iPSC source cells' originating tissue. IF labeling revealed terminally differentiated cell types derived from the three germ layers under spontaneous conditions. These results demonstrate robust generation of iPSCs from an accessible AEC source, which are comparable to gold-standard fibroblast-derived iPSCs.

### Example 2

This example demonstrates CRISPR-Cas9 gene manipulation in AEC-derived iPSCs.

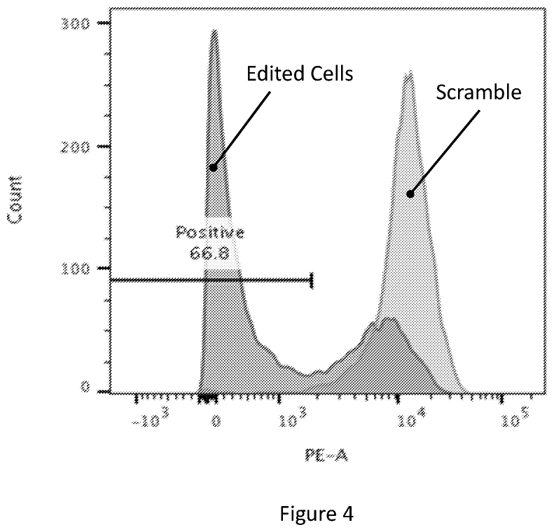

Pre-assembled Cas9 protein with guide CRISPR oligonucleotides to form ribonucleoprotein complexes (RNPs) is a potent approach for achieving highly efficient, safer, and faster gene editing than the conventional plasmid-based methods. To test the capacity of the AEC-derived iPSCs for gene editing, RNP nucleofection was employed of the AEC-derived iPSCs for both gene knock-out by non-homologous end joining (NHEJ) and gene modification by homology-directed repair (HDR). For NHEJ capacity, dual CRISPR guides were designed to target epithelial cell adhesion molecule (EPCAM, aka CD326) in AEC-derived iPSCs, achieving knock-out of over 66% by flow cytometry (FIG. 4). EPCAM knock-out cells displayed decreased cell-cell contacts, eventually leading to cell death, and by cell counting, cell proliferation was significantly inhibited in knock-out cells compared to scramble control cells. HDR editing was achieved through knock-in of GFP on the beta-actin gene (REF plasmid). Three days post-transfection, GFP positive cells were detected three days post transfection in RNP plus HDR template co-transfected cells but not RNP or HDR template alone. GFP knock-in efficiency was up to 1.17% by flow cytometry. Flow-sorted GFP⁺ clones were further expanded and screened for correct HDR by PCR with specific primers flanking each homology arm. Correctly edited GFP⁺ cells were retained their pluripotency characteristics.

### Example 3

This example describes differentiation of iPSCs to airway epithelial cells.

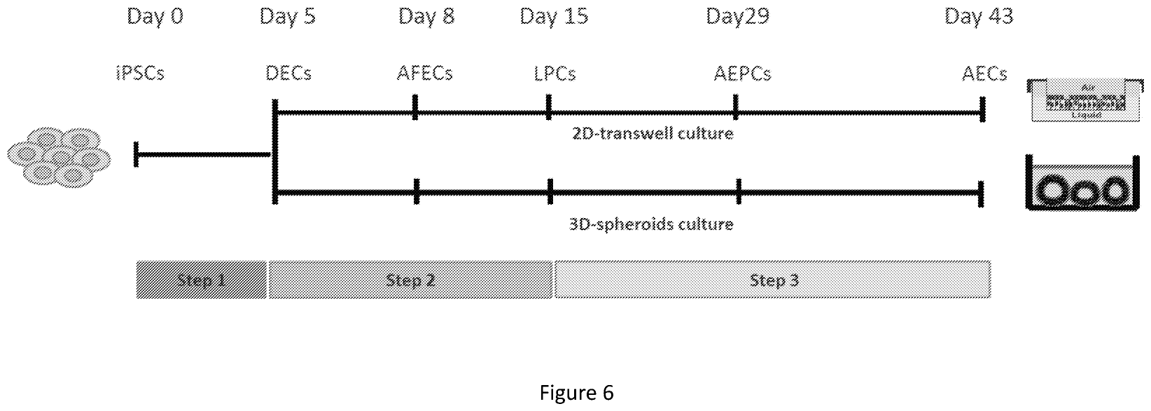

Airway epithelium from the primary AEC-derived iPSCs described herein was generated using both 2-dimensional(2D) transwell-based air-liquid interface (ALI) and 3-dimensional(3D) lung spheroid differentiation protocols (see, for example, McCauley et al., “Derivation of Epithelial-Only Airway Organoids from Human Pluripotent Stem Cells”, Curr Protoc Stem Cell Biol. 4 May 2018) (FIG. 6).

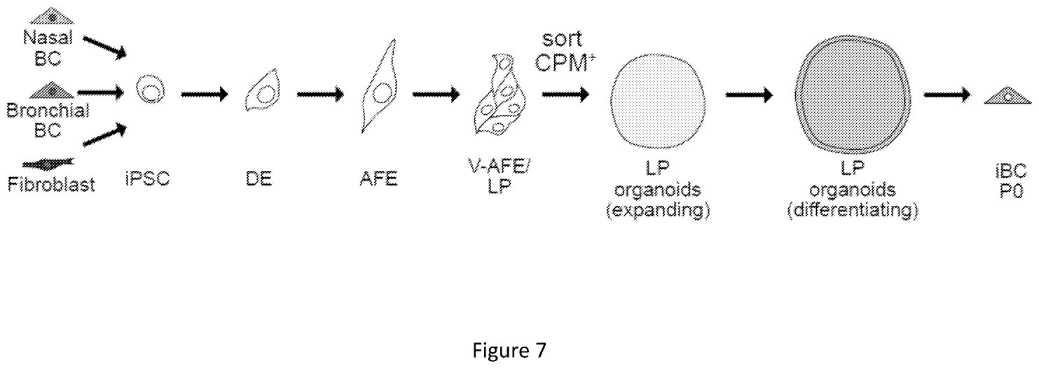

To mimic ventralized anterior foregut endoderm-derived epithelium development in the embryo, stepwise differentiation protocols in 2D were adapted to monolayer culture (FIG. 7). iPSCs were directed to differentiate into definitive endoderm using methods disclosed herein (see, for example, Paragraph E under Methods). Pluripotency markers (OCT3/4 and SOX2) were lost during generation of definitive endoderm (DE), which was marked by induction of SOX/7 and FOXA2 expression) and >95% CD184(CXCR4)+/CD117(c-KIT)+co-expression by flow cytometry. DE were then directed, using either 2D or 3D methods, to form anterior foregut endoderm (AFE) (see paragraph E under Methods), indicated by further induction of FOXA2 and reappearance of SOX2 expression. The continued expression of the latter markers as well as appearance of transcriptional regulator NKX2.1 signaled the subsequent transition from AFE to Ventralized-AFE, containing the earliest lung progenitor (LP) cells.

### Example 4

This example describes differentiation of lung progenitor cells into iBCs

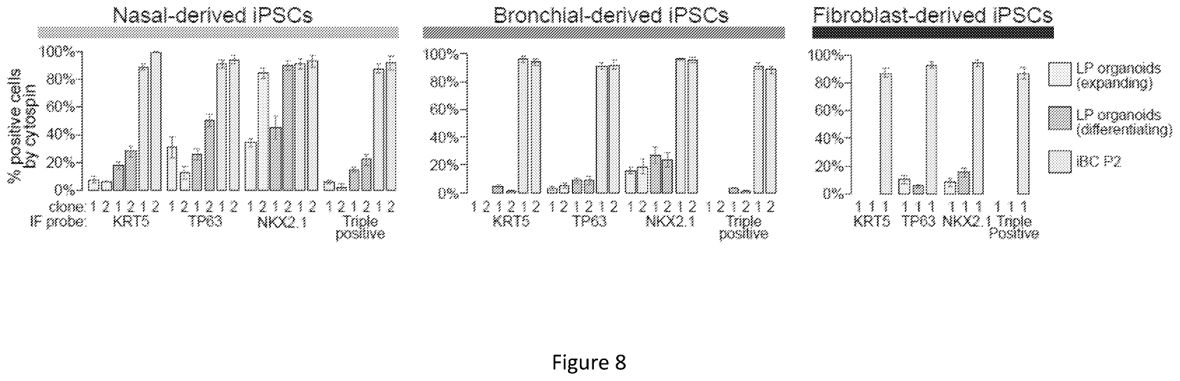

LP populations were enriched via Carboxypeptidase M (CPM)+ immuno-fluorescence activated cell sorting (FACS), and the progenitors were further specified in Matrigel-based 3D organoid culture via expansion and differentiation stages (FIG. 8) (See, for example, paragraphs E, F, and G, under METHODS). While most LP expanding organoid cells expressed NKX2.1, KRT5 and TP63 expression began only in a small subset of cells during the LP differentiating organoid stage. To isolate, expand and further specify these progenitor BCs, dissociated LP differentiating organoids were transferred to feeder fibroblast co-culture with dual SMAD and RHO kinase (ROCK) inhibition, conditions preferred by primary BCs (see, for example, paragraph G under METHODS). Serial passaging resulted in a flourishing homogeneous population of iBCs that were nearly 100% triple-positive for KRT5/TP63/NKX2.1 (FIG. 8). These iBCs were void of the mesenchymal marker VIM protein expression, a hallmark of potential contaminating alternative lineages, present in both LP organoid stages. iBCs retained triple-positive expression, tight colony morphology and expansion capacity across at least seven passages and with cryopreservation. Importantly, high quality iBCs were generated from at least 5 iPSC clones, derived from different tissue sources including non-airway fibroblast controls, demonstrating the robustness of the disclosed iBC generation protocol.

### Example 5

This example demonstrates that proteasomal signatures of differentiating organoids precede extensive iBC specification

Since differentiating LP organoids, but not expanding LP organoids, were capable of facilitating functional iBC production downstream, single cell sequencing was used to assess the specification processes that occurred during the progression from LP expanding organoids through iBC expansion. Organoid cells were stratified into basal-like and less basal cells according to mean expression of published basal cell signatures. Both expanding and differentiating organoids contained proliferating and non-proliferating basal-like cells, less basal cells and a few specialized cell types. As anticipated, differentiating organoids contained 15-fold more cells in the ciliated/PNEC cluster than expanding organoids, whereas the remaining broad categories were similarly represented in both organoid stages. Notably, despite comparable relative proportions of both less basal and basal-like cells, differentiating organoids had significantly increased mean expression of basal cell signatures relative to expanding organoids. This increased basal character supports additional basal cell specification that occurs in differentiating organoids, potentially contributing to their ability to generate quality iBCs.

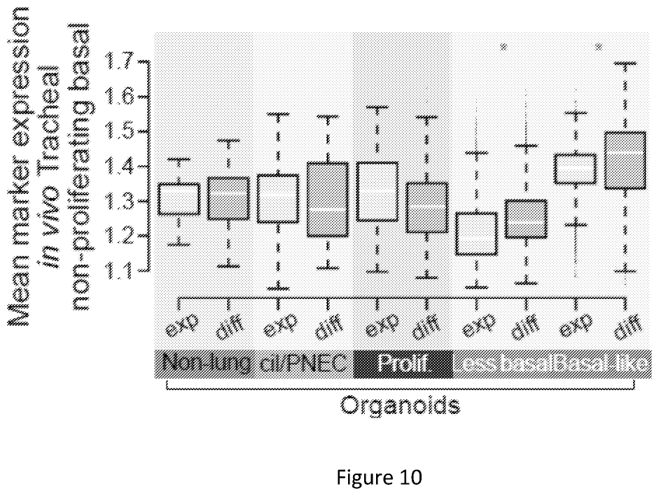

Beyond diagnostic marker expression, iBC samples enhanced basal cell character relative to organoids' basal-like cells. iBC samples clustered into six cell states (FIG. 10), most of which had comparable or higher mean expression of published basal cell signatures relative to organoid basal-like cells. Further, agnostic differential expression revealed more than 2000 DEGs were significantly upregulated in at least one iBC state relative to the basal-like cells of organoids, including a core set of 277 genes that were significantly upregulated across at least 5 iBC states. This core iBC signature included KRT5 and several S100 genes as well as DEGs enriched for energy production, mRNA processing and stability, cell cycle checkpoints and myriad signaling cascades known to be crucial for development, redefining the vast functions gained post-organoid culture. Many distinguishing features among the iBC states were also more highly expressed in iBCs relative to organoids' basal-like cells. Classic proliferation markers (e.g. TOP2A, MKI67) and a host of DEGs enriched for cell cycle related functions were significantly upregulated in iBCs' proliferating basal state relative to the proliferating basal cells in organoids. Compared to other iBC states, the quiescent iBC state most highly expressed TP63, basal cell adhesion molecule (BCAM), the caveolins, and several WNT and NOTCH ligands, and these key regulators were also largely absent in the basal-like cells of organoids. The squam-ish iBC state likely represented basal cells slightly differentiated toward a hillock or squamous phenotype, described in vivo by us and others, including upregulation relative to both other iBCs and organoids' basal-like cells of KRT4 and KRT13, envelope proteins EVPL and SPRR1B, and desmosome components like desmoplakin (DSP) and PERP. Similarly, the club-ish state most highly expressed markers of lower airway secretory differentiation including TGFB receptor II, surfactant proteins (SFTPA2, SFTPB), epithelial membrane composition regulator EMP2, and detoxifiers CYP4B1 and AGR2. Finally, the adhesion focused state significantly upregulated a suite of genes focused on cell-cell contacts including numerous integrins, jagged NOTCH ligands, and catenins. Together, these data indicate iBCs acquire substantial additional basal cell character after the organoid stages.

In contrast, a portion of the DEGs distinguishing each iBC state from the remaining iBCs were comparably or more highly expressed by the basal-like cells in organoids. Whereas the proliferating, quiescent and squam-ish states only had roughly 30% of their distinctive features expressed comparably in organoids, organoids highly expressed more than half of the club-ish, adhesion focused and XXX states' DEGs, suggesting that the basal-like cells in organoids may more closely resemble these latter states. Organoids' basal-like cells and iBCs' club-ish state both highly expressed proteins involved in cell defense like MUC1, secretoglobin 1A1, SERPINB1, tissue factor and members of the complement cascade. Growth factor regulators SMAD2, FGFR3 and VEGFA were among those co-expressed in organoids' basal-like cells and iBCs' adhesion focused state. The only NOTCH receptor expressed in iBCs is found in the squam-ish state and also organoids' basal-like cells, implicating a putative NOTCH competition between NOTCH3 in the squam-ish and the various NOTCH ligands specifically expressed by the quiescent and adhesion focused states, which is absent in organoids. Finally, iBCs' tiny XXX state uniquely expressed protease inhibitors alpha-1 antitrypsin and tissue factor pathway inhibitor, transcriptional regulator SOX4, inflammatory factors (ITIH2, CXCL3, IL6ST) and ion transporters associated with ionocytes CFTR and ATP6V0B. These shared expression profiles suggest that the basal-like cells in organoids are largely secretory leaning, with mixed specification signals relative to the more defined iBC states.

To investigate which processes and/or cell states occur in differentiating organoids (capable of seeding precursor iBCs) that were missing in expanding organoids (putatively too primitive for iBC generation), a direct differential expression analysis between the differentiating and expanding organoids for each cell population was conducted. The proliferating basal cells in differentiating organoids stood out, exhibiting the most distinct signature from the expanding organoid counterpart with over 10-fold more unique DEGs than any other basal-like cell cluster. The majority of this unique signature had strong enrichment for cilium assembly, organization, and maintenance, including several early ciliating markers (e.g. FOXN4, DEUP1, E2F7, STIL, PLK4, CDC20B, CCNO etc.), as well as numerous diagnostic ciliated cell genes (e.g. FOXJ1, RFX3, etc.). Since DAPT was present in the culture media only during the differentiating organoid stage, this ciliating signature may indicate the gamma secretase-driven induction of ciliogenesis occurs primarily in this proliferating basal cell population. Further, most differentiating organoid cell populations carried a core enhanced stress signature relative to those in the expanding organoids or iBCs. This stress signature likely reflects the cumulative burden of 4 weeks culture in the same vessel, and may contribute a key transitional state preceding the generation of iBCs.

Differentiating organoids also highly expressed a heavily proteasomal profile with enrichments for apoptosis, cellular response to heat stress, organelle biogenesis and maintenance, as well as mRNA processing including several keratins ubiquitously expressed by the iBCs. Whereas this proteasomal module was upregulated in the differentiating organoids sample relative to the expanding organoids, most iBC populations also highly expressed these genes. In fact, this module was significantly upregulated across cell states in iBC samples relative to basal-like cells from differentiating organoids, implying the latter as a putative precursor for iBC generation. Together these data suggest a model where differentiating organoids enhance proteasomal and stress response signatures to enable an iBC-precursor environment, before further specification in feeder co-culture.

### Example 6

This example demonstrates that iBCs possess a stem-ish ground state phenotype relative to their primary BC counterparts

To assess the authenticity of the iBC populations, a comparison of morphology, marker protein expression patterns, growth rates and transcriptomic profiles to primary basal cells expanded from the same donor that generated the iPSCs used for iBC regeneration was performed. In co-culture with fibroblast feeders, BCs and iBCs displayed indistinguishable tight “island” colonies. While both BCs and iBCs express KRT5 in almost all cells, IF labeling of dissociated primary BCs revealed a large size distribution, where only the subset of smallest cells co-expressed nuclear TP63 expression. This likely reflects the primary BCs' tendency to differentiate and diverge from stemness in prolonged culture. In contrast, dissociated iBCs were uniformly small, with the vast majority co-expressing nuclear TP63, mimicking the subset of primary basal cells predicted to be the most potent. This homogeneity was echoed in enhanced proliferative capacity of iBCs relative to primary BCs, suggesting that our iBCs represent an amplification of the optimal primary basal cell population.

At the single cell transcriptional level, while all basal cell states contained cells from both primary BCs and iBCs, iBC samples contained more cells in the proliferating and quiescent states (57-59%) relative to primary BCs (38%). Regardless of cell state, most cells from the primary BC sample expressed significantly higher levels of a core defensive secretory/stress signature including stressed basal cell markers KRT14 and KRT6A, club cell marker SCGB1A1, interleukin receptors IL20RB and IL1RN, all three MEW class I antigen-presenting molecules and IL33. In contrast, most cells from iBC samples upregulated genes involved in adhesion like cadherin 1 (CDH1), EPCAM, desmocollin 2 (DSC2), and beta-catenin, or other developmental processes like NKX2.1. Beyond these core differences, primary BCs in multiple states had increased expression of various keratins (KRT15, KRT5, KRT23), cytokines (CXCL6, CXCL8, CXCL16, IL18), transmembrane mucins (MUC16, MUC20) and other secretory defensive proteins (BPIFA2, BPIFB1, CP, C3), while iBCs in multiple states had increased expression of other regulators including FOXA2, FOXP1, SOX11, SOX6 and SOX4. These contrasting signatures may indicate molecular memory of inflammation and environmental stress in the primary BCs, which is replaced by a ground state multipotency in the iBCs.

Consistent with this inflammatory memory in primary BCs, TSLP was upregulated in primary BCs within the quiescent state exclusively, while caveolin, KRT4 and WNT6 were uniquely upregulated in iBCs' quiescent cells. Finally, the club-ish state had the most unique differences between primary BCs and iBCs, where primary BCs' club-ish population was preparing for mucus production by upregulating transcription factors FOXC1 and SPDEF, as well as glycosylation machinery (e.g. GALNT7), SCGB3A1 and MUC5AC. In contrast, iBCs' club-ish population had elevated expression of surfactant proteins (SFTPA2, SFTPB) and a suite of genes enriched for metabolic, structural and gene expression functions including TGF-beta ligand BMP4, microtubule/cytoskeleton organizers ezrin and centrin 2, and critical cell cycle regulators like geminin and TOP2A. Notably, many of these genes uniquely upregulated in the club-ish state of iBCs are also ubiquitously expressed by the proliferating population across samples, implying that iBCs' club-ish state is more proliferative than the club-ish state of primary BCs.

Together, these data suggest that primary BCs reside in a more inflammation-primed state, where initial differentiation is poised for mucus production while iBCs appear to be more suspended in the quiescent state, exhibiting more multipotent qualities with a tendency toward the lower airway surfactant-rich secretory differentiation.

### Example 7

This example demonstrates that iALI cultures resemble primary cultures and in vivo epithelium.

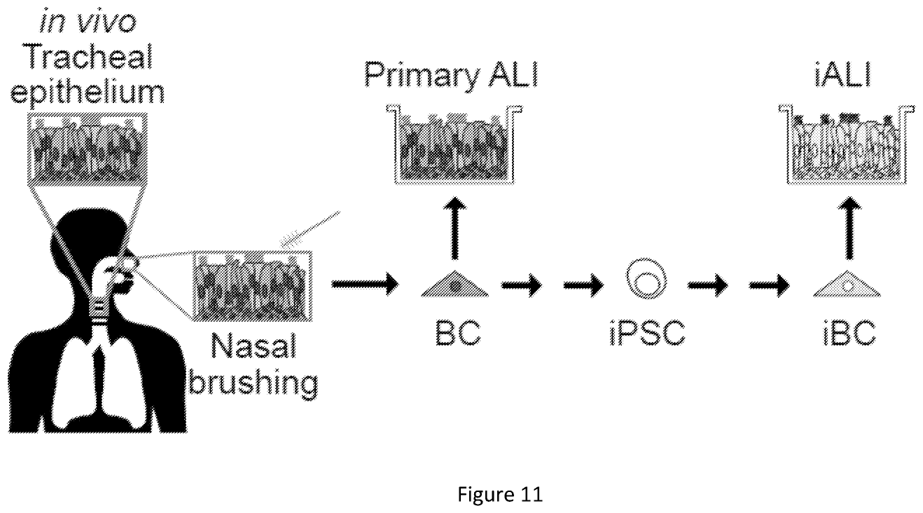

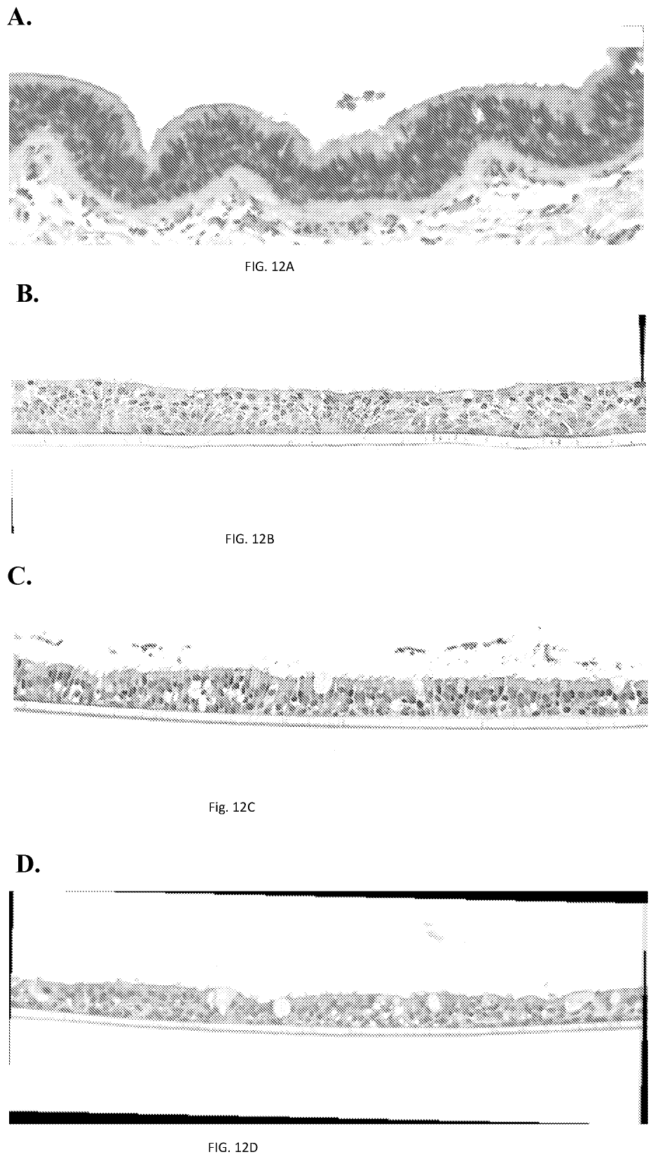

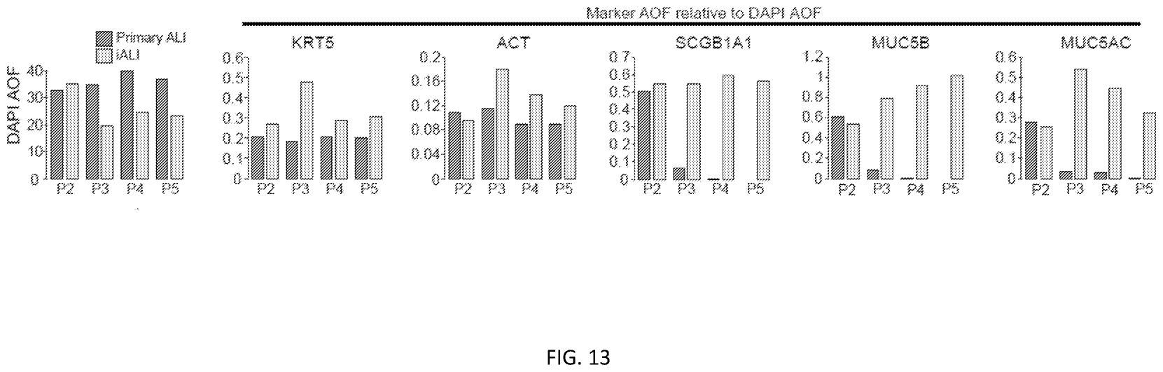

As stem cells, airway BCs have both proliferative and differentiation capacity. The ability of the iBCs to produce pseudostratified airway epithelium at air-liquid-interface (ALI) was examined, comparing this to the ALI cultures produced in vitro by primary BCs and the in vivo proximal airway epithelium from the same donor (FIG. 11). Wholemount IF labeling demonstrated highly consistent well-differentiated ALI and induced ALI (iALI) cultures including mature mucus (MUC5AC+) and ciliated (ACT+) cells with tight apical junctions (ECAD+). Histological sections illustrated classic pseudostratified epithelia with BCs along the basement membrane (FIGS. 12A-12D). iBCs produced fully differentiated epithelia across at least 7 passages, as evidenced by basal, mucus and ciliated cell counts (FIG. 13).

### Example 8

This example further demonstrates the differentiation potential of iBCs produced using methods disclosed herein.

To test the differentiation potential of the iBCs described herein, epithelia generated from primary airway basal cells and iBCs was compared via a standard transwell-based ALI differentiation protocol. As early as 10 days post airlift, both cultures displayed ciliary beating by phase-contrast microscopy. IF labeling of Day 21 ALI cultures revealed a highly consistent pseudostratified epithelium with abundant MUC5AC⁺ mucus secreting cells, SCGB1A1⁺ club cells, and ACT⁺ multiciliated cells on the apical surface while KRT5 and TP63 double positive basal cells lined the basal membrane. Top-down IF staining revealed intact epithelial junction by E-Cadherin. Notably, both primary and iBC-derived epithelia lacked the thick mesenchymal under layer seen with 2D AEC differentiation protocol, suggesting iBC isolation from 3D-AEC differentiation with Dual-SMAD inhibitors selectively against these mesenchymal progenitors. Together, these data demonstrate the first generation of primary-comparable fully potent airway basal cells and airway epithelia from iPSCs.

While various embodiments of the present invention have been described in detail, it is apparent that modifications and adaptations of those embodiments will occur to those skilled in the art. It is to be expressly understood, however, that such modifications and adaptations are within the scope of the present invention, as set forth in the following exemplary claims.

We claim:

1. A method of producing induced basal cells (iBCs), comprising:
a. obtaining induced pluripotent stem cells (iPSCs); and
b. directing generation of iBCs from the iPSCs, wherein the directing lacks genetic manipulation.

2. The method of claim 1, wherein the step of directing results in production of a homogenous population of iBCs in which at least 80% of the iBCs are KRT5+, TP63+, and NKX2.1+.

3. The method of claim 1 or 2, wherein the step of obtaining induced pluripotent stem cells (iPSCs)comprises:
a. obtaining a sample of primary cells (PCs);
b. culturing and expanding the PCs;
c. transfecting the PCs with RNA-based reprogramming factors; and,
d. identifying and purifying iPSCs.

4. The method of any one of claims 1-3, wherein the step of directing generation of iBCs comprises:
a. culturing the iPSCs to form lung organoids from the iPSCs;
b. differentiating the lung organoids to form airway epithelial spheroids comprising airway epithelial cells;
c. dissociating the airway epithelial spheroids and culturing the airway epithelial cells with gamma-irradiated fibroblasts, wherein the culture conditions comprise Duel-SMAD inhibition and, optionally, an inhibitor of rho-associated coiled coil containing kinase (ROCK), thereby forming iBCs.

5. The method of claim 4, comprising 3D organoid culture.

6. The method of claim 4 or 5, wherein the step of culturing iPSCs to form lung organoids comprises:
culturing the iPSCs under conditions such that they form definitive endoderm (DE);
culturing the DE under conditions that direct differentiation of the DE into anterior foregut endoderm; (AFE)
culturing the AFE under conditions that direct differentiation of the AFE into lung progenitor cells;
culturing the lung progenitor cells under conditions that direct the lung progenitor cells to form lung organoids.

7. The method of claim 6, wherein prior to culturing the lung progenitor cells, the population of lung progenitor cells is enriched.

8. The method of claim 7, wherein enriching the population of lung progenitor cells comprises antibody-bases cell sorting.

9. The method of claim 8, wherein the antibody is an anti-carboxypeptidase antibody.

10. The method of any one of claims 6-9, wherein the DE and/or the AFE are cultured using conditions comprising at least one extracellular matrix protein.

11. The method of any one of claims 6-10, wherein the DE and/or the AFE are cultured using conditions comprising one or more inhibitors selected from the group consisting of an inhibitor of a bone morphogenic protein (BMP) pathway, an inhibitor of transforming growth factor beta (TGF-β)/Activin/NODAL/pathway, and an inhibitor of glycogen synthase kinase-3 (GSK3).

12. The method of any one of claims 3-11, wherein the PCs are obtained by taking a tissue or cell sample from an individual.

13. The method of claim 12, wherein the tissue or cell sample is obtained by brushing a cell surface, lavage, or by surgical excision.

14. The method of any one of claims 3-13, wherein the PCs are airway epithelial cells (AECs).

15. The method of claim 14, wherein the AECs are nasal epithelial cells or bronchial airway epithelial cells.

16. The method of any one of claims 1-15, wherein the iPSCs are human iPSCs.

17. The method of any one of claims 1-16, wherein the genome of the iPSC has been genetically modified.

18. An induced basal cell (iBC) prepared using the method of any one of claims 1-17.

19. A method of producing an epithelial tissue, comprising culturing the iBC of claim 18 in an air-liquid interface culture.

20. An epithelial tissue produced using the method of claim 19.

21. A method of treating an individual in need of such treatment, comprising administering the iBC of claim 18 or the epithelial tissue of claim 20 to the individual.

22. The method of claim 21, wherein the iBC or the epithelial tissue is administered to treat the individual for a respiratory disease.

23. The method of claim 21 or 22, wherein administration comprises transplanting the iBC or the epithelial tissue into the subject's epithelium.

24. The method of any one of claims 21-23, wherein the epithelium is nasal epithelium, oral epithelium, pharyngeal epithelium, laryngeal epithelium, tracheal epithelium, bronchial epithelium, and/or lung epithelium.

25. Use of the method of any one of claims 1-17, in preparing an induced basal cell (iBC).

26. Use of the iBC of claim 18 in preparing an epithelial tissue.

27. Use of the method of any one of claims 1-17, the iBC of claim 18, or the epithelial tissue of claim 20, in preparing a primary cell or tissue-based model of a disease.

28. Use of the method of any one of claims 1-17, the iBC of claim 18, or the epithelial tissue of claim 20, in studying a biological response to a compound or an environmental stimulus.

29. Use of the method of any one of claims 1-17, the iBC of claim 18, or the epithelial tissue of claim 20, in the preparation of a medicament or therapeutic composition for treating a respiratory illness.

30. Use of the method of any one of claims 1-17, the iBC of claim 18, or the epithelial tissue of claim 20, in identifying a therapeutic compound.

31. The use of claim 30, where in the compound is for the treatment of a respiratory disease.
